(19) United States
(12) Patent Application Publication
(10) Pub. No.: US 2024/0159706 A1
(43) Pub. Date: May 16, 2024

(54) MALDI-MS METHOD, PHOTO-SENSITIVE MALDI MATRIX COMPOSITE AND PHOTO-CAGED MALDI MATRIX COMPOUND FOR USE IN SAID METHOD AND RESPECTIVE USES

(71) Applicant: SIRIUS FINE CHEMICALS SICHEM GMBH, Bremen (DE)

(72) Inventors: Carsten HOPF, Mannheim (DE); Hartmut GILLANDT, Bremen (DE)

(73) Assignee: SIRIUS FINE CHEMICALS SICHEM GMBH, Bremen (DE)

(21) Appl. No.: 18/546,137

(22) PCT Filed: Feb. 11, 2022
(86) PCT No.: PCT/EP2022/053399

§ 371 Date: Aug. 11, 2023

(30) Foreign Application Priority Data
Feb. 11, 2021 (EP) 21156663.3

(51) Int. Cl. G01N 27/64; G01N 1/36; H01J 49/16

(52) U.S. Cl. CPC G01N 27/64; G01N 1/36; H01J 49/164

(57) ABSTRACT

The present invention relates to a Photo-caged MALDI Matrix Compound for use in a method of matrix-assisted laser desorption/ionization mass spectrometry, as well as to a sprayable liquid composition and a Photo-sensitive MALDI Matrix Composite, both comprising said Photo-caged MALDI Matrix Compound. Moreover, the present invention pertains to a matrix-assisted laser desorption/ionization mass spectrometry method involving the Photo-sensitive MALDI Matrix Composite and the Photo-caged MALDI Matrix Compound. Under a further aspect, the present invention pertains to a method of identifying for a given type of analyte and predetermined vacuum conditions a suitable Photo-caged MALDI Matrix Compound.

### CROSS-REFERENCE TO RELATED APPLICATIONS

This application is a § 371 national stage entry of International Application No. PCT/EP2022/053399, filed Feb. 11, 2022, which claims priority to European Patent Application No. 21156663.3, filed on Feb. 11, 2021, the entire contents of which are incorporated herein by reference.

The present invention relates to a Photo-caged MALDI Matrix Compound for use in a method of matrix-assisted laser desorption/ionization mass spectrometry, as well as to a sprayable liquid composition and a Photo-sensitive MALDI Matrix Composite, both comprising said Photo-caged MALDI Matrix Compound.

Moreover, the present invention pertains to a matrix-assisted laser desorption/ionization mass spectrometry method involving the Photo-sensitive MALDI Matrix Composite and the Photo-caged MALDI Matrix Compound. Under a further aspect, the present invention pertains to a method of identifying, for a given type of analyte and predetermined vacuum conditions, a suitable Photo-caged MALDI Matrix Compound.

Matrix-assisted laser desorption/ionization (MALDI) mass spectrometry (MS) is an ionization technique that uses a laser energy-absorbing matrix to create ions from large molecules with minimal fragmentation.

MALDI-MS has been used for the analysis of larger molecules, including biomolecules, e.g. biopolymers such as nucleic acids, proteins, peptides or polysaccharides, or of other organic molecules, including polymers, dendrimers or certain macromolecules, in particular such larger molecules which tend to be fragile and to fragment when ionized by other, less gentle ionization methods. Today, MALDI-MS and its variants have developed into extremely valuable analytical tools for clinical and pharmaceutical research.

The conventional MALDI-MS method usually comprises three steps, i.e. (i) mixing a sample to be analysed (usually referred to as the “analyte”) with a suitable matrix material (also referred to as “MALDI matrix compound”), thereby embedding the analyte in the matrix material, ideally including co-crystallizing the analyte with the matrix material, and applying the matrix material with the embedded analyte (together also referred to herein as a “MALDI matrix composite”) to an object slide, (ii) irradiating the matrix material with the embedded analyte with a pulsed laser to trigger ablation and desorption thereof from the object slide, usually including creation of a gas phase, and (iii) ionizing the analyte molecules, presumably by protonation or deprotonation in the hot plume of ablated gases. Subsequently, the ionized analyte molecules, in the gas phase, are provided to the inlet of a mass spectrometer.

The expression “embedding” an analyte in a MALDI matrix compound (or the respective expression “embedded”, respectively), more specifically in a Photo-caged MALDI Matrix Compound as disclosed herein, generally refers to and includes any method or process (or the result of such method or process, respectively) suitable for combining the analyte with the MALDI matrix compound (viz. the Photo-caged MALDI Matrix Compound as defined below) to prepare a MALDI matrix composite (more specifically a Photo-sensitive MALDI Matrix Composite as disclosed herein) which can be used in a matrix-assisted laser desorption/ionization mass spectrometry method, as is known and customary in the technical field. A particular example of such method or process is the direct tissue analysis on tissue samples (cf. e.g. S. A. Schwartz et al., J. Mass Spectrom. 38 (2003) 699-708, doi: 10.1002/jms.505) wherein peptide and protein MS signals can effectively be desorbed directly from cells and tissues, e.g. from mouse brain. In this method, a MALDI matrix compound is directly applied onto a tissue surface for dissolving an analyte of interest, e.g. to extract a protein from its native environment on the tissue, and subsequently embedding (or incorporating) the extracted analyte in (a matrix of) the MALDI matrix compound (thereby ideally co-crystallizing the extracted analyte with the MALDI matrix compound) to form a MALDI matrix composite. Other examples of such known methods or processes which are also comprised by the expression “embedding” as used herein comprise the method of whole-cell MS (cf. e.g. B. Munteanu et al. in “Advances in MALDI and Laser-Induced Soft Ionization Mass Spectrometry”, 249-262 Springer International Publishing, Switzerland 2016 (R. Cramer ed.)), the dried droplet or bottom layer method, the direct analysis method, the premix (or volume or one-layer) method, the two-layer (or seed-layer) method and the on-probe method (for all of the foregoing cf. e.g. G. M. Toh-Boyo et al. Anal. Chem. 84, 22 (2012) 9971-9980).

The expression “combining a MALDI matrix compound (specifically a Photo-caged MALDI Matrix Compound; or a sprayable liquid composition thereof), with one or more analytes” is used synonymously herein with the expression “embedding an analyte in a MALDI matrix compound (specifically a Photo-caged MALDI Matrix Compound; or a sprayable liquid composition thereof”).

MALDI-MS is increasingly used today in high-throughput screening processes where larger molecular libraries need to be analysed within relatively short time periods. This application sets high demands on automatization techniques, robust test methods and analysis of results.

MALDI mass spectrometry imaging (MALD-MSI) is a more specific application of MALDI-MS, i.e. a technique for visualization of the spatial distribution of small molecular and macromolecular biomolecules in tissue sections. MALDI-MSI is often used in pharmaceutical analytics and facilitates an in-depth analysis of drug compounds, metabolites, adjuvants and contaminants in a tissue sample. In related analytical methods, about 200.000 measuring points need to be scanned with a step size of about 20 μm over a period of one hour. It is planned for future applications to further reduce the distance between measuring points by a factor of ten, which means an increase of measuring points by a factor of 100 and to measure entire (clinical) cohorts consisting of 100 and more such samples, which also means an increase of measuring points by a factor of 10. For realizing such ambitious goals, MALDI-MS or MALDI-MSI measuring cycle times of up to 72 hours or more will be required.

Since MALDI techniques usually involve applying a vacuum, a MALDI matrix compound suitable for future high-performance MALDI-MS or MALDI-MSI analytical methods, e.g. in processes for serial imaging of multiple tissue sections—like entire clinical cohorts—with high spatial resolution, will need to be stable under such vacuum conditions over extended time periods, e.g. for time periods of about 72 hours or more, as explained above, and not prematurely evaporate. However, many MALDI matrix compounds known today do usually not possess the required long-term stability against evaporation under vacuum.

In order to address the challenges that arise from the need of keeping larger numbers of samples ready for testing in a MALDI-MS or MALDI-MSI analytical method for extended time periods, the method of atmospheric pressure MALDI (“AP-MALDI”) was recently introduced (cf. e.g. S. N. Jackson et al., J. Am. Soc. Mass Spectrom. 29, 7 (2018) 1463-1472; or C. Keller et al., Front. Plant Sci. 2018).

In the light of the above, there is still a need for an alternative or improved MALDI-MS or MALDI MSI method and/or for reagents for use in such method, where said method should preferably allow analysing a larger number of samples, ideally as a high-throughput method, with high quality of the analytical results.

Correspondingly, it was a primary object of the present invention to provide a suitable MALDI matrix compound which would provide for increased vacuum stability, e.g. to allow extended measuring cycles in MALDI-MS or MALDI-MSI methods, and a sprayable liquid composition comprising such MALDI matrix compound.

It was another object of the present invention to provide a MALDI matrix composite with increased stability against evaporation upon exposure to a vacuum.

It was a further object of the present invention to provide a MALDI-MS or MALDI-MSI method which would make use of a MALDI matrix composite and/or a MALDI matrix composite with increased stability against evaporation upon exposure to a vacuum, e.g. to allow for extended measuring cycles.

An additional object of the present invention pertained to providing a method for identifying—for a given type of analyte and predetermined vacuum conditions—a suitable MALDI matrix compound which would provide for increased vacuum stability of a resulting MALDI matrix composite

It has now been found that the primary object and other objects of the present invention can be accomplished by a Photo-caged MALDI Matrix Compound, preferably for use in a method of matrix-assisted laser desorption/ionization mass spectrometry, comprising or consisting of a Photoremovable Protecting Group Moiety B which is covalently bonded to an organic MALDI Matrix Compound Moiety A, wherein
the organic MALDI Matrix Compound Moiety A is a moiety selected from the group consisting of
   a moiety of formula I-1

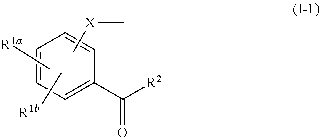

wherein
R^{1a} and R^{1b} are both independently of each other selected from the group consisting of hydrogen, hydroxy, carboxy, methyl and methoxy,
R² is selected from the group consisting of hydroxy and methyl and
X is selected from the group consisting of oxygen, —N(H)—, —N(CH₃)— and —N(C₂H₅)—, and preferably is oxygen;
wherein the moiety of formula I-1 is attached (or covalently bonded) to the Photoremovable Protecting Group Moiety B via the free bond to the right of group X;
a moiety of formula I-2

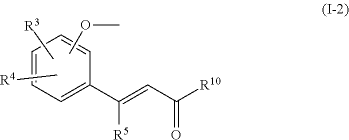

wherein
R³ and R⁴ are independently of each other selected from the group consisting of hydrogen, hydroxy, halogen and methoxy and
R⁵ is selected from the group consisting of hydroxy, methyl and cyano and
R¹⁰ is selected from the group consisting of hydroxy and methyl,
wherein the moiety of formula I-2 is attached (or covalently bonded) to the Photoremovable Protecting Group Moiety B via the free bond to the right of the oxygen atom attached to the phenyl ring;
a moiety of formula I-3

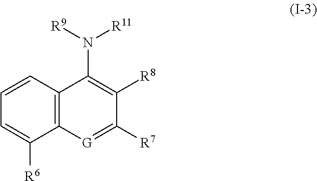

wherein
G is selected from the group consisting of nitrogen and C—H;
R⁶ is selected from the group consisting of hydrogen and amino,
R⁷ and R⁸ are independently of each other selected from the group consisting of hydrogen, amino and methyl, or together with the carbon atoms to which they are bonded, form a saturated or unsaturated five or six-membered ring,
R⁹ is selected from the group consisting of hydrogen, phenyl, branched or unbranched, saturated alkyl having 1 to 4 carbon atoms and branched or unbranched, saturated aminoalkyl having 1 to 4 carbon atoms, and
R¹¹ is a bond or hydrogen,
wherein the moiety of formula I-3 is covalently bonded to the Photoremovable Protecting Group Moiety B via a free bond of a nitrogen atom present in the moiety of formula I-3;
and
   a moiety which is selected from the group consisting of the moieties of formula I-4, formula I-5 and formula I-6:

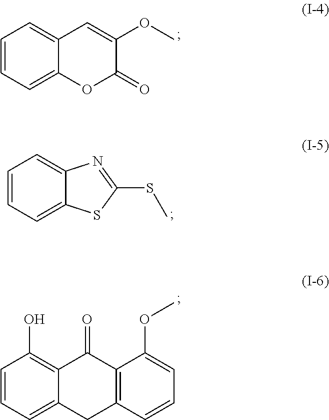 [SMILES: COc1cc2ccccc2oc1=O ; CSc1nc2ccccc2s1 ; COc1cccc2c1C(=O)c1c(O)cccc1C2]

wherein the moieties of formula I-4 and of formula I-6 are attached to the Photoremovable Protecting Group Moiety B via the respective free bond of an oxygen atom, and wherein the moiety of formula I-5 is attached to the Photoremovable Protecting Group Moiety B via the free bond of a sulfur atom;
and wherein
   the Photoremovable Protecting Group Moiety B is a moiety of formula II

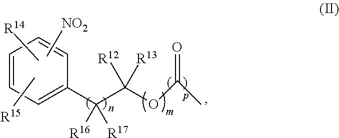

wherein
R¹² is selected from the group consisting of hydrogen and branched or unbranched, saturated alkyl with 1 to 4 carbon atoms, preferably consisting of hydrogen and methyl;
R¹³ is selected from the group consisting of hydrogen, branched or unbranched, saturated alkyl with 1 to 4 carbon atoms (preferably methyl) and carboxy;
R¹⁴ and R¹⁵ are both attached to the phenyl ring, wherein
   R¹⁴ and R¹⁵ are both independently of each other selected from the group consisting of hydrogen, methoxy, carboxy, nitro and —O(CH₂)_{I}COOH, wherein I is an integer in the range from 1 to 3; preferably selected from the group consisting of hydrogen, methoxy and —O(CH₂)_{I}COOH, wherein I is an integer in the range from 1 to 3;
   or
   R¹⁴ and R¹⁵ together form an acetal group having two oxygen atoms attached to adjacent carbon atoms of the phenyl ring and having 1 to 3 carbon atoms outside the phenyl ring;
   wherein preferably
      R¹⁴ is selected from the group consisting of hydrogen, methoxy, carboxy, nitro and —O(CH₂)_{I}COOH, wherein I is an integer in the range from 1 to 3; and
      R¹⁵ is selected from the group consisting of hydrogen and methoxy;
R¹⁶ and R¹⁷ are both hydrogen or, together with the carbon atom to which they are bonded, form a carbonyl group; and
m, n, and p are independently of each other 0 or 1,
   wherein preferably n is 0 and wherein preferably m is 0 when p is 0;
and wherein the Photoremovable Protecting Group Moiety B of formula II is attached (covalently bonded) to an organic MALDI Matrix Compound Moiety A via the free bond next to the letter “p” shown in formula II above.

The invention as well as preferred variants and preferred combinations of parameters, properties and elements thereof are defined in the appended claims. Preferred aspects, details, modifications and advantages of the present invention are also defined and explained in the following description and in the examples shown below.

In the context of the present invention (and of all aspects of the present disclosure as described herein), the meaning of the term “matrix-assisted laser desorption/ionization mass spectrometry”, in accordance with the usual meaning in the field, comprises the methods of MALDI-MS, MALDI-MSI and “MALDI-2” as are generally known in the field. “MALDI-2” is a more sensitive MALDI-MS method involving laser-induced post-ionization of the analyte and/or the MALDI matrix compound, to enhance mass spectrometry imaging of numerous classes of biomolecules, as e.g. described by J. Soltwisch et al. in SCIENCE, Vol. 348, Issue 6231 (2015), pp. 211-215; or by J. Soltwisch et al. in Anal. Chem. 92, 13 (2020) pp. 8697-8703.

In the context of the present invention (and of all aspects of the present disclosure as described herein), the term “photoremovable protecting group”, in accordance with the usual meaning in the field, means a chemical group which can be attached to a chemical (usually organic) molecule and can be (preferably selectively) removed again from said chemical (usually organic) molecule with electromagnetic radiation of a wavelength in the range of from ≥100 nm to ≤15 μm, or with electromagnetic radiation of the preferred wavelength ranges as further defined herein. Common synonyms for the term “photoremovable protecting group” are “photosensitive protecting group”, “photocleavable protecting group” or “photoreleasable protecting group”. A variety of photoremovable protecting groups is known in the prior art, e.g. from P Klan et al. in “Chemical Reviews” 113 (2013) pp. 119-191, and literature cited therein, as well as from A. Y. Vorobev in Computational and Structural Biotechnology Journal 18 (2020) pp. 27-34, or from the “CRC Handbook of Organic Photochemistry and Photobiology”, in particular its chapter on “Photolabile Protecting Groups in organic Synthesis” CRC Press 3^{rd} ed. 2012 (or previous editions of this book).

The skilled person is aware that a substance in order to be generally suited as a MALDI matrix compound should preferably show one or more typical properties, comprising:
(a) a pronounced absorption in the region of commonly used UV or IR laser wavelengths, e.g. by the presence of two or more than two conjugated double bonds in the chemical structure of the MALDI matrix compound;
(b) at best cause minimal matrix background signals and adducts with analytes (“chemical noise”), es-pecially in the low mass range (typically m/z<500), thus enabling visualization of the spatial distribution of small molecules;
(c) an ability to interact with analyte molecules, ideally an ability of forming co-crystals with analyte molecules;
(d) an ability to effectively ionize analyte molecules, resulting in protonated ions in positive ion mode or deprotonated ions in negative ion mode.

The Photo-caged MALDI Matrix Compound according to the present invention (and of all aspects of the present disclosure as described herein) is photo-cleavable, preferably at the covalent bond (or at a covalent bond) between its moieties A and B (as explained above), upon irradiation with suitable radiation of a wavelength in the range of from ≥100 nm to ≤15 μm (or a preferred wavelength range as described herein). Without wishing to be bound by theory, it is assumed that, upon cleavage of the photo-cleavable Photo-caged MALDI Matrix Compound, the MALDI Matrix Compound Moiety A releases a MALDI matrix compound, i.e. a compound which is suitable to act as a matrix compound in a MALDI-MS or MALDI-MSI method. In certain cases, the Photo-caged MALDI Matrix Compound may itself represent or act as a MALDI matrix compound (i.e. is suitable to act as a matrix compound in a MALDI-MS or MALDI-MSI method).

The Photo-caged MALDI Matrix Compound according to the present invention (and according to all aspects of the present disclosure as described herein) has a higher molecular mass and a lower vapor pressure (at 23° C.) than the corresponding MALDI matrix compound which may be released from it by photo-cleavage. This has the effect that said (released) MALDI matrix compound is less stable against evaporation when exposed to a vacuum than the corresponding Photo-caged MALDI Matrix Compound from which it can be released by photo-cleavage. A Photo-sensitive MALDI Matrix Composite according to the present invention (or according to each and any aspect of the present disclosure as described herein) is therefore particularly stable for extended time periods under vacuum conditions as are usually applied in methods of matrix-assisted laser desorption/ionization mass spectrometry like e.g. high-throughput MALDI-MS or MALDI-MSI methods.

Where the Photoremovable Protecting Group Moiety B is a moiety of formula II and a substituent R¹⁴ or R¹⁵ in said moiety of formula II is or comprises a carboxy group (“—COOH”, including a carboxy group of the residue “—O(CH₂)_{I}COOH”), such carboxy group comprises in the meaning of its definition the undissociated carboxy group, as well as the respective carboxylate anion and an alkali metal salt of said carboxylate anion, where the alkali metal is preferably selected from the group consisting of lithium, sodium and potassium. In a preferred variant of the present invention, said carboxy group is present in a moiety of formula II as undissociated carboxy group (“—COOH”).

Where the Photoremovable Protecting Group Moiety B is a moiety of formula II and the substituents R¹⁴ and R¹⁵ together form an acetal group having two oxygen atoms attached to adjacent carbon atoms of the phenyl ring and having 1 to 3 carbon atoms outside the phenyl ring, the meaning of said acetal group comprises acetal groups and ketal groups, in particular acetonides (isopropylidene ketals). Formula II-1 shown below provides an example where the substituents R¹⁴ and R¹⁵ together form an acetal group having two oxygen atoms attached to adjacent carbon atoms of the phenyl ring and having 3 carbon atoms outside the phenyl ring:

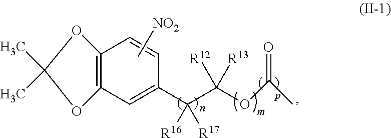

Where the organic MALDI Matrix Compound Moiety A of the Photo-caged MALDI Matrix Compound according to the present invention (or according to any aspect of the present disclosure as described herein) is a moiety of formula I-1, it is preferably derived from a corresponding compound which, upon common chemical modification, provides a group “X” to which the Photoremovable Protecting Group Moiety B can be attached. Preferred corresponding compounds from which moieties of formula I-1 are preferably derived are selected from the group consisting of 2,5-dihydroxybenzoic acid (also known as DHB or gentisic acid), 2,5-dihydroxyacetophenone (2,5-DHAP), 2,6-dihydroxyacetophenone (2,6-DHAP), 2,4,6-trihydroxyaceto-phenone, 2-(methylamino)benzoic acid (also known as COOH—NHMe) and 2,5-dihydroxyterephthalic acid.

Where the organic MALDI Matrix Compound Moiety A of the Photo-caged MALDI Matrix Compound according to the present invention (or according to any aspect of the present disclosure as described herein) is a moiety of formula I-2, it is preferably derived from a corresponding compound which, upon common chemical modification, provides a respective oxygen atom to which the Photoremovable Protecting Group Moiety B can be attached. Preferred corresponding compounds from which moieties of formula I-2 are preferably derived are selected from the group consisting of alpha-cyano-4-hydroxycinnamic acid (also known as HCCA or CHCA), 4-chloro-alpha-cyanocinnamic acid, sinapinic acid (also known as trans-3,5-dimethoxy-4-hydroxy cinnamic acid or SA), ferulic acid (also known as trans-3-methoxy-4-hydroxy cinnamic acid or FA) and (E)-4-(2,5-dihydroxyphenyl) but-3-en-2-one (also known as 2,5-cDHA).

Where the organic MALDI Matrix Compound Moiety A of the Photo-caged MALDI Matrix Compound according to the present invention (or according to any aspect of the present disclosure as described herein) is a moiety of formula I-3, it is preferably derived from a corresponding compound, which, upon common chemical modification, provides a respective nitrogen atom to which the Photoremovable Protecting Group Moiety B can be attached. Preferred corresponding compounds from which moieties of formula I-3 are preferably derived are selected from the group consisting of 2-aminoquinoline, 3-aminoquinoline, 4-aminoquinoline, 8-aminoquinoline, N-(1-naphthyl)ethylene diamine dihydrochloride (also known as NEDC), 1,5-diaminonaphthalene (also known as 1,5-DAN) and N-phenyl-2-naphthylamine (also known as PNA).

Where the organic MALDI Matrix Compound Moiety A of the Photo-caged MALDI Matrix Compound according to the present invention is a moiety of formula I-4, it is preferably derived from 3-aminoquinoline. Where the organic MALDI Matrix Compound Moiety A of the Photo-caged MALDI Matrix Compound according to the present invention is a moiety of formula I-5, it is preferably derived from 2-mercaptobenzothiazol. Where the organic MALDI Matrix Compound Moiety A of the Photo-caged MALDI Matrix Compound according to the present invention is a moiety of formula I-6, it is preferably derived from dithranol.

Preferred Photoremovable Protecting Group Moieties B of formula II for the purposes of the present invention or for the purposes of further aspects as disclosed herein are selected from the group consisting of 4,5-dimethoxy-2-nitrobenzyl (also known as DMNB), alpha-carboxy-2-nitrobenzyl (also known as CNB), 1-(2-nitrophenyl)ethyl (also known as NPE), 5-carboxymethoxy-2-nitrobenzyl (also known as CMNB), 1-(4,5-dimethoxy-2-nitrophenyl)ethyl (also known as DMNPE) and 2-nitroveratryloxycarbonyl (also known as NVOC). The 4,5-dimethoxy-2-nitrobenzyl group is particularly preferred as a Photoremovable Protecting Group Moiety B of formula II for the purposes of the present invention.

It has been found in own experiments that a nitrophenyl group, preferably a nitrophenyl group of formula II as defined above or below, is particularly suited as a Photoremovable Protecting Group Moiety B of a Photo-caged MALDI Matrix Compound for use in a Photo-sensitive MALDI Matrix Composite according to the present invention, or according to further aspects disclosed herein. Photo-caged MALDI Matrix Compounds comprising a nitrophenyl group of formula II as a Photoremovable Protecting Group Moiety B were found to show (i) good or very good solubility in common organic solvents or mixtures of common organic solvents with water, (ii) good or very good formation of crystals, (iii) good or very good stability against evaporation when exposed to a vacuum (in reduced pressure ranges as are common in MALDI-MS methods) and (iv) indication of cleavage and release of the corresponding MALDI matrix compound upon irradiation with radiation of a wavelength in the range of from ≥100 nm to ≤15 μm, preferably in the range of from ≥150 nm to ≤750 nm and particularly preferably of a wavelength which is usually applied in common MALDI mass spectrometers, i.e. a wavelength in the range of from ≥300 nm to ≤400 nm, preferably of 355 nm (e.g. of a frequency-tripled neodymium-doped yttrium aluminum garnet laser) and/or of 337 nm (e.g. of a nitrogen laser).

Preferred is a Photo-caged MALDI Matrix Compound according to the present invention as described herein (or a Photo-caged MALDI Matrix Compound according to the present invention as described herein as preferred) wherein
the organic MALDI Matrix Compound Moiety A is a moiety selected from the group consisting of
   a moiety of formula I-1 as defined above (or a moiety of formula I-1 as described herein as preferred),
   a moiety of formula I-2 as defined above (or a moiety of formula I-2 as described herein as preferred),
and
   a moiety of formula I-3a

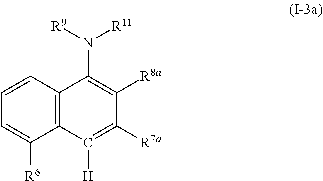

wherein
R⁶, R⁹ and R^{8a} have the meanings as defined above for the moiety of formula I-3,
R^{7a} and R^{8a} are independently of each other selected from the group consisting of hydrogen and methyl,
and
wherein the moiety of formula I-3a is covalently bonded to the Photoremovable Protecting Group Moiety B via a free bond of a nitrogen atom present in the moiety I-3a.

In own experiments it has also been found that a Photo-caged MALDI Matrix Compound for use in a Photo-sensitive MALDI Matrix Composite according to the present invention or according to further aspects as disclosed herein is preferred, wherein a moiety of formula I-1 (as defined above or below) forms the MALDI Matrix Compound Moiety A and a nitrophenyl group of formula II (as defined above or below) forms the Photoremovable Protecting Group Moiety B. For such preferred Photo-caged MALDI Matrix Compounds, the above-stated beneficial properties (i) to (iv) were found to be particularly pronounced.

Preferred is therefore a Photo-caged MALDI Matrix Compound according to the present invention as defined herein (or a Photo-caged MALDI Matrix Compound according to the present invention as described herein as preferred) or according to further aspects as disclosed herein, comprising as Photoremovable Protecting Group Moiety B a nitrophenyl group of formula II (as defined above) and as organic MALDI Matrix Compound Moiety A a moiety of formula I-1 (as defined above).

Also preferred is a Photo-caged MALDI Matrix Compound according to the present invention as described herein (or a Photo-caged MALDI Matrix Compound according to the present invention as described herein as preferred), selected from the group consisting of:
a compound of formula III-1

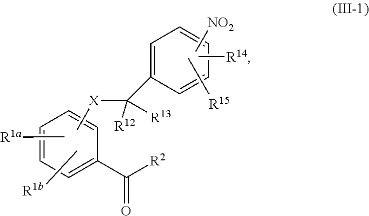

wherein
   R^{1a}, R^{1b}, R² and X have the meanings (or preferred meanings) as defined above for the moiety of formula I-1, wherein preferably R^{1a} and R^{1b} are both independently of each other selected from the group consisting of hydrogen and methoxy and/or X is oxygen
      and
   R¹², R¹³, R¹⁴ and R¹⁵ have the meanings (or preferred meanings) as defined above for the moiety of formula II, wherein preferably R¹⁴ is selected from the group consisting of hydrogen, methoxy, carboxy and nitro;
a compound of formula III-2

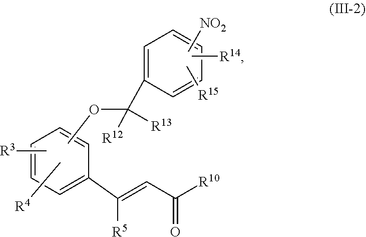

wherein
   R³, R⁴, R⁵ and R¹⁰ have the meanings as defined in above for the moiety of formula I-2 and
   R¹², R¹³, R¹⁴ and R¹⁵ have the meanings (or preferred meanings) as defined above for the moiety of formula II, wherein preferably R¹⁴ is selected from the group consisting of hydrogen, methoxy, carboxy and nitro;
and
   a compound of formula III-3

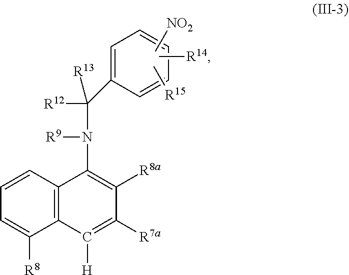

wherein
   R⁶ and R⁹ have the meanings as defined above for the moiety of formula I-3,
   R¹², R¹³, R¹⁴ and R¹⁵ have the meanings (or preferred meanings) as defined above for the moiety of formula II,
and
   R^{7a} and R^{8a} are independently of each other selected from the group consisting of hydrogen and methyl.

A Photo-caged MALDI Matrix Compound of formula III-1 as defined here above preferably results from a combination of a MALDI Matrix Compound Moiety A which is a moiety of formula I-1 as described above (or as described above as preferred), with a Photoremovable Protecting Group Moiety B which is a moiety of formula II as described above (or as described above as preferred), wherein the moiety of formula I-1 is attached (covalently bonded) to the moiety of formula II via the free bond to the right of group “X” shown in formula I-1. In the moiety of formula II attached to the moiety of formula I-1 in this case, m,n and p are all “0”.

A Photo-caged MALDI Matrix Compound of formula III-2 as defined here above preferably results from a combination of a MALDI Matrix Compound Moiety A which is a moiety of formula I-2 as described above (or as described above as preferred), with a Photoremovable Protecting Group Moiety B which is a moiety of formula II as described above (or as described above as preferred), wherein the moiety of formula I-2 is attached (covalently bonded) to the moiety of formula II via the free bond to the right of the oxygen atom attached to the phenyl ring shown in formula I-2. In the moiety of formula II attached to the moiety of formula 1-2 in this case, m,n and p are all “0”.

A Photo-caged MALDI Matrix Compound of formula III-3 as described here above preferably results from a combination of a MALDI Matrix Compound Moiety A which is a moiety of formula I-3a as described above (or as described above as preferred), with a Photoremovable Protecting Group Moiety B which is a moiety of formula II as described above (or as described above as preferred), wherein the moiety of formula I-3a is attached (covalently bonded) to the moiety of formula II via the free bond provided by R¹¹ as shown in formula I-3a. In the moiety of formula II attached to the moiety of formula I-3a in this case, m,n and p are all “0”.

Accordingly, in variants of the present invention (and of all aspects of the present disclosure as described herein) a Photo-caged MALDI Matrix Compound according to the present invention as defined herein (or a Photo-caged MALDI Matrix Compound according to the present invention as described herein as preferred) is preferably selected from the group consisting of 1-(5-((4,5-dimethoxy-2-nitrobenzyl)oxy)-2-hy-droxyphenyl)ethan-1-one, N¹-(4,5-dimethoxy-2-nitrobenzyl)naphthalene-1,5-diamine, 3-((4,5-dimethoxy-2-nitrobenzyl)oxy)-2H-chromen-2-one, 1-(2-((4,5-dimethoxy-2-nitrobenzyl)oxy)-6-hydroxyphenyl)ethan-1-one, 2-((4,5-dimethoxy-2-nitrobenzyl)thio)benzo[d]thiazole, 1-((4,5-dimethoxy-2-nitrobenzyl)oxy)-8-hydroxyanthracen-9(10H)-one, 4-((3-acetyl-4-hydroxyphenoxy)methyl)-3-nitrobenzoic acid, 4-(4-(1-(3-acetyl-4-hydroxyphenoxy)ethyl)-2-methoxy-5-nitrophenoxy)butanoic acid, 3-acetyl-4-hydroxyphenyl (4,5-di-methoxy-2-nitrobenzyl) carbonate, and mixtures thereof.

In a more specific variant of the present invention is preferred a Photo-caged MALDI Matrix Compound according to the present invention as described herein (or a Photo-caged MALDI Matrix Compound according to the present invention as described herein as preferred), which is a compound of formula 1.1

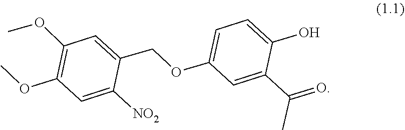 [SMILES: COc1cc(COc2ccc(O)c(C(C)=O)c2)c([N+](=O)[O-])cc1OC]

The compound of formula 1.1 as shown above is also known as 1-(5-((4,5-dimethoxy-2-nitrobenzyl)oxy)-2-hydroxyphenyl)ethan-1-one and is also referred to herein as “DMNB-2,5-DHAP”. This compound has shown in own experiments a combination of beneficial properties which makes it particularly suited as Photo-caged MALDI Matrix Compound for use in a method of matrix-assisted laser desorption/ionization mass spectrometry.

The Photo-caged MALDI Matrix Compound according to the present invention as described herein can be prepared by methods known in the art or by methods analogous to said known methods.

The present invention also pertains to a sprayable liquid composition comprising a Photo-caged MALDI Matrix Compound according to the present invention as defined herein (or a Photo-caged MALDI Matrix Compound as defined herein as preferred), wherein preferably the sprayable liquid composition comprises or is a solution or dispersion of said Photo-caged MALDI Matrix Compound in a solvent or in a mixture of two or more solvents.

Generally, all aspects of the present invention discussed herein in the context of the Photo-caged MALDI Matrix Compound according to the present invention as described herein apply mutatis mutandis to the sprayable liquid composition comprising a Photo-caged MALDI Matrix Compound according to the present invention as described herein, and vice versa.

Where the sprayable liquid composition according to the present invention comprises water as solvent, a sprayable liquid composition is preferred which has a pH in the range of from 2 to 8, preferably of from 4 to 8. A sprayable liquid composition according to the present invention comprising water as solvent is preferred for a Photo-caged MALDI Matrix Compound (according to the present invention) comprising one or more carboxy groups.

A sprayable liquid composition comprising a Photo-caged MALDI Matrix Compound according to the present invention is a formulation which is particularly suitable for applying the Photo-caged MALDI Matrix Compound according to the present invention to the surface of a tissue sample, e.g. for performing a direct tissue MALDI MS or MALDI-MSI method.

The present invention further pertains to a matrix-assisted laser desorption/ionization mass spectrometry (MALDI-MS) method, comprising the following steps:
S0) providing or preparing a Photo-caged MALDI Matrix Compound according to the present invention as described herein (or a Photo-caged MALDI Matrix Compound according to the present invention as described herein as preferred), or a sprayable liquid composition according to the present invention as described herein (or a sprayable liquid composition according to the present invention as described herein as preferred),
S1) combining the Photo-caged MALDI Matrix Compound or a sprayable liquid composition thereof as provided or prepared in step S0) with one or more analytes so that a Photo-sensitive MALDI Matrix Composite results,
S2) irradiating, in one, two, or more steps, the Photo-sensitive MALDI Matrix Composite resulting from step S1) so that
   (i) the Photo-caged MALDI Matrix Compound is photo-cleaved,
   and
   (ii) one or more of said analytes are desorbed/ionized, and
S3) analyzing one or more of said analytes desorbed/ionized in step S2) by mass spectrometry.

Generally, all aspects of the present invention discussed herein in the context of the Photo-caged MALDI Matrix Compound according to the present invention as described herein and/or to the sprayable liquid composition comprising a Photo-caged MALDI Matrix Compound according to the present invention as described herein apply mutatis mutandis to the matrix-assisted laser desorption/ionization mass spectrometry method according to the present invention as described herein, and vice versa.

Combining the Photo-caged MALDI Matrix Compound or a sprayable liquid composition thereof with one or more analytes in step S1) can be done by any method or process suitable for preparing a MALDI matrix composite, more specifically a Photo-sensitive MALDI Matrix Composite as disclosed herein, which can be used in a matrix-assisted laser desorption/ionization mass spectrometry method, as is known and customary in the technical field and as is explained in more detail above.

Preferred is a matrix-assisted laser desorption/ionization mass spectrometry method according to the present invention as described herein (or a matrix-assisted laser desorption/ionization mass spectrometry method according to the present invention as described herein as preferred), wherein the Photo-sensitive MALDI Matrix Composite resulting from step S1) is kept under vacuum for a time period of at least one hour before it is irradiated in step S2),
wherein preferably
   the Photo-sensitive MALDI Matrix Composite resulting from step S1) is kept under vacuum for a time period of at least 8 hours, more preferably for a time period of at least 24 hours, even more preferably for a time period of at least 36 hours and yet even more preferably for a time period of between 36 and 72 hours, before it is irradiated in step S2);
   or
   the Photo-sensitive MALDI Matrix Composite resulting from step S1) is kept under vacuum for a time period of from ≥36 hrs to ≤72 hrs, preferably of from ≥48 hrs to ≤96 hrs, more preferably of from 72 hrs to ≤120 hrs, before it is irradiated in step S2);
and/or
   the Photo-sensitive MALDI Matrix Composite resulting from step S1) is kept under vacuum at a pressure of 3300 Pa or below, preferably of 5 Pa or below, more preferably of 0.1 Pa or below and even more preferably of 10-4 Pa or below, yet even more preferably of 10-5 Pa or below, before it is irradiated in step S2).

As is explained above, stability of a MALDI matrix compound, viz. a Photo-caged MALDI Matrix Compound according to the present invention, against undesired premature evaporation is of particular value where a vacuum atmosphere needs to be applied in MALDI-MS or MALDI-MSI methods. A Photo-caged MALDI Matrix Compound as described herein has a higher molecular mass and a lower vapor pressure (at 23° C.) than the MALDI matrix compound which can be released from it by photo-cleavage. According to the present invention, a MALDI matrix compound can therefore be stabilized against evaporation when exposed to a vacuum by converting it into a corresponding Photo-caged MALDI Matrix Compound. By this method according to the present invention (or according to all aspects of the present disclosure as described herein), MALDI matrix compounds with a very low vapor pressure may be made available to MALDI-MS methods for the first time, or MALDI matrix compounds with a low vapor pressure can be made available to MALDI-MSI methods, where stability against evaporation upon exposure to a vacuum for extended time periods is critical, for the first time.

Further preferred is a matrix-assisted laser desorption/ionization mass spectrometry method according to the present invention as described herein (or a matrix-assisted laser desorption/ionization mass spectrometry method according to the present invention as described herein as preferred), wherein
step S2) and/or step S3) is conducted under vacuum, preferably at a pressure of 3300 Pa or below, more preferably of 5 Pa or below, even more preferably of 0.1 Pa or below and yet even more preferably of 10-4 Pa or below;
and/or
irradiating the Photo-sensitive MALDI Matrix Composite in step S2) comprises irradiating with a laser source, preferably a pulsed laser source, more preferably a modulated pulsed laser source, wherein preferably the wavelength applied for irradiating is in the range of from ≥150 nm to ≤700 nm, more preferably of from ≥250 nm to ≤400 nm and even more preferably the wavelength applied for irradiating comprises or is the wavelength of 355 nm.

Where in step S2), irradiating the Photo-sensitive MALDI Matrix Composite in step S2) comprises irradiating with a laser source, said irradiating with a radiation of a suitable wavelength (as defined above or below) preferably has the effect that the Photo-caged MALDI Matrix Compound comprised by the Photo-sensitive MALDI Matrix Composite, which is likewise exposed to said radiation, is photo-cleaved by the impact of the said radiation, preferably at the site of the covalent bonding between the Photoremovable Protecting Group Moiety B and the organic MALDI Matrix Compound Moiety A, so that a MALDI matrix compound is released. The MALDI matrix compound so released preferably corresponds to the compound from which the organic MALDI Matrix Compound Moiety A was derived.

Laser sources suitable for providing the radiation for photo-cleaving the Photo-caged MALDI Matrix Compound according to the present invention (or according to all aspects of the present disclosure as described herein), preferably photo-cleaving at the covalent bond between its moieties A and B, comprise common lasers known in the art. Such lasers include UV lasers, in particular nitrogen lasers (operating at a wavelength of 337 nm), frequency-tripled and quadrupled neodymium-doped yttrium aluminum garnet lasers (Nd:YAG lasers, operating at a wavelength of 355 nm or 266 nm, respectively); infrared (IR) lasers, in particular erbium-doped yttrium aluminum garnet lasers (Er:YAG lasers, operating at a wavelength of 2.94 μm), mid-IR optical parametric oscillators and carbon dioxide lasers (operating at a wavelength of 10.6 μm). Diode lasers are also suited for the purpose explained above.

UV lasers, in particular nitrogen lasers (operating at a wavelength of 337 nm), frequency-tripled and quadrupled neodymium-doped yttrium aluminum garnet lasers (Nd:YAG lasers, operating at a wavelength of 355 nm or 266 nm, respectively) are preferred for the purposes of the present invention where a Photo-caged MALDI Matrix Compound according to the present invention is used. Particularly suited examples of lasers for providing the radiation for photo-cleaving the Photo-caged MALDI Matrix Compound of the present invention are modulated Bruker Smartbeam™ Lasers as described e.g. by A. Holle et al. in “J. Mass Spectrom.” 41 (2006) 705-716, DOI: 10.1002/jms.1041.

Preferred is furthermore a matrix-assisted laser desorption/ionization mass spectrometry method according to the present invention as described herein (or a matrix-assisted laser desorption/ionization mass spectrometry method according to the present invention as described herein as preferred), wherein
the method is a high-throughput method analyzing 64 or more samples per hour, wherein preferably irradiating the Photo-sensitive MALDI Matrix Composite in step S2) comprises successively irradiating all of said samples with the same laser source;
and/or
the matrix-assisted laser desorption/ionization mass spectrometry method is a mass spectrometry imaging method, wherein preferably in step S1) the Photo-caged MALDI Matrix Compound is combined with one or more analytes which are present in a tissue sample by contacting a liquid composition of the Photo-caged MALDI Matrix Compound (preferably a liquid composition according to the present invention as defined above) with said tissue sample, preferably to co-crystallize the one or more analytes with the Photo-caged MALDI Matrix Compound.

Also preferred is a matrix-assisted laser desorption/ionization mass spectrometry method according to the present invention as described herein (or a matrix-assisted laser desorption/ionization mass spectrometry method according to the present invention as described herein as preferred), wherein step S1) comprises
co-crystallizing a Photo-caged MALDI Matrix Compound according to the present invention as described herein (or a Photo-caged MALDI Matrix Compound according to the present invention as described herein as preferred) with the one or at least one of the more than one analyte;
and/or
contacting a liquid composition of a Photo-caged MALDI Matrix Compound (preferably a liquid composition according to the present invention as defined above or as defined above as preferred) with a tissue sample so that one or more analytes from the tissue sample are combined, and preferably subsequently co-crystallized, with the Photo-caged MALDI Matrix Compound.

A matrix-assisted laser desorption/ionization mass spectrometry method according to the present invention, wherein step S1) comprises contacting a liquid composition of a Photo-caged MALDI Matrix Compound or with a liquid composition according to the present invention with a tissue sample (e.g. a sample of brain tissue from a mammal) so that one or more analytes (e.g. one or more proteins) from the tissue sample are combined, and preferably subsequently co-crystallized, with the Photo-caged MALDI Matrix Compound, is particularly suited as a MALDI-MSI method of direct tissue analysis, as described above.

A matrix-assisted laser desorption/ionization mass spectrometry method according to the present invention as described herein (or a matrix-assisted laser desorption/ionization mass spectrometry method according to the present invention as described herein as preferred) is preferred, wherein
the one or at least one of the more than one analyte is selected from the group consisting of proteins, peptides, lipids, nucleic acids, polysaccharides, glycopeptides, organometallic compounds and organic polymers, wherein preferably the molar mass of the analyte is in the range of from ≥100 Da to ≤200000 Da, more preferably of from ≥100 Da to ≤50000 Da and even more preferably of from ≥100 Da to ≤15000 Da;
and/or
the total mass ratio of the total mass of Photo-caged MALDI Matrix Compound present in the Photo-sensitive MALDI Matrix Composite: total mass of analyte present in the Photo-sensitive MALDI Matrix Composite is in the range of from ≥100:1 to ≤20000:1, preferably of ≥500:1 to ≤10000:1.

Where the total mass ratio of Photo-caged MALDI Matrix Compound: analyte present in the Photo-sensitive MALDI Matrix Composite is defined herein, the term “total mass” refers to the total mass of the Photo-caged MALDI Matrix Compound or the analyte, respectively, both as present in the Photo-sensitive MALDI Matrix Composite, not their molar masses. For example, where the respective total mass ratio is 200:1, this means that, where the Photo-sensitive MALDI Matrix Composite comprises 1 mg of the one or more analytes, the Photo-sensitive MALDI Matrix Composite then comprises 200 mg of the Photo-caged MALDI Matrix Compound.

The present invention further pertains to a Photo-sensitive MALDI Matrix Composite for use in a method of matrix-assisted laser desorption/ionization mass spectrometry, comprising or consisting of
C1) a matrix, completely or partially constituted by a Photo-caged MALDI Matrix Compound according to the present invention as described herein (or a Photo-caged MALDI Matrix Compound according to the present invention as described herein as preferred), and embedded in said matrix
C2) one or more than one analyte, to be analyzed in the method of matrix-assisted laser desorption/ionization mass spectrometry.

Generally, all aspects of the present invention discussed herein in the context of the Photo-caged MALDI Matrix Compound according to the present invention as described herein and/or to the sprayable liquid composition comprising a Photo-caged MALDI Matrix Compound according to the present invention as described herein and/or to the matrix-assisted laser desorption/ionization mass spectrometry method according to the present invention as described herein apply mutatis mutandis to the Photo-sensitive MALDI Matrix Composite according to the present invention as described herein, and vice versa.

In component C1) of the Photo-sensitive MALDI Matrix Composite according to the present invention as defined above (or according to all aspects of the present disclosure as described herein), the matrix may be completely or partially constituted by a Photo-caged MALDI Matrix Compound. For example, the matrix can be completely constituted by a Photo-caged MALDI Matrix Compound (which is the preferred option) or the matrix can be partially constituted by a Photo-caged MALDI Matrix Compound and partially by a MALDI matrix compound as is known in the art. In the latter case, the matrix can in particular be partially constituted by a Photo-caged MALDI Matrix Compound and partially by a MALDI matrix compound which comprises an equal organic MALDI Matrix Compound Moiety A as is also part of the Photo-caged MALDI Matrix Compound constituting the same matrix.

Preferred is a Photo-sensitive MALDI Matrix Composite according to the present invention as described herein (or a Photo-sensitive MALDI Matrix Composite according to the present invention as described herein as preferred) is preferred, wherein
the one or at least one of the more than one analyte is selected from the group consisting of proteins, peptides, lipids, nucleic acids, polysaccharides, glycopeptides, organometallic compounds and organic polymers, wherein preferably the molar mass of the analyte is in the range of from ≥100 Da to ≤200000 Da, more preferably of from ≥100 Da to ≤50000 Da and even more preferably of from ≥100 Da to ≤15000 Da;
and/or
the total mass ratio of the total mass of Photo-caged MALDI Matrix Compound present in the Photo-sensitive MALDI Matrix Composite: total mass of analyte present in the Photo-sensitive MALDI Matrix Composite is in the range of from ≥100:1 to ≤20000:1, preferably of ≥500:1 to ≤10000:1.

The present invention further pertains to the use of a Photo-caged MALDI Matrix Compound according to the present invention as described herein (or a Photo-sensitive MALDI Matrix Composite according to the present invention as described herein as preferred)
as matrix compound in a method of matrix-assisted laser desorption/ionization mass spectrometry
and/or
for preparing a Photo-sensitive MALDI Matrix Composite according to the present invention as described herein (or a Photo-sensitive MALDI Matrix Composite according to the present invention as described herein as preferred).

Generally, all aspects of the present invention discussed herein in the context of the Photo-caged MALDI Matrix Compound according to the present invention as described herein and/or to the sprayable liquid composition comprising a Photo-caged MALDI Matrix Compound according to the present invention as described herein and/or to the matrix-assisted laser desorption/ionization mass spectrometry method according to the present invention as described herein and/or to the Photo-sensitive MALDI Matrix Composite according to the present invention as described herein apply mutatis mutandis to the use of a Photo-caged MALDI Matrix Compound according to the present invention as described herein, and vice versa.

Under a different angle, the present invention also pertains to a method of identifying for a given type of analyte and predetermined vacuum conditions a suitable Photo-caged MALDI Matrix Compound according to the present invention as described herein (or a Photo-caged MALDI Matrix Compound according to the present invention as described herein as preferred), comprising the steps of
providing for said type of analyte a suitable uncaged MALDI matrix compound comprising the organic MALDI Matrix Compound Moiety A but not comprising the Photoremovable Protecting Group Moiety B,
preparing or providing one or more derivatives of said suitable uncaged MALDI matrix compound, wherein each of said derivatives is a different Photo-caged MALDI Matrix Compound comprising a different Photoremovable Protecting Group Moiety B which is covalently bonded to the organic MALDI Matrix Compound Moiety A present in said uncaged MALDI matrix compound,
assessing the dependency of MALDI analysis results from predetermined vacuum conditions for the combinations of said type of analyte with both said suitable uncaged MALDI matrix compound and said one or more derivatives of said suitable uncaged MALDI matrix compound,
and
identifying the suitable Photo-caged MALDI Matrix Compound comprising a Photoremovable Protecting Group Moiety B which is covalently bonded to an organic MALDI Matrix Compound Moiety A by comparing the results of the assessments.

Generally, all aspects of the present invention discussed herein in the context of the Photo-caged MALDI Matrix Compound according to the present invention as described herein and/or to the sprayable liquid composition comprising a Photo-caged MALDI Matrix Compound according to the present invention as described herein and/or to the matrix-assisted laser desorption/ionization mass spectrometry method according to the present invention as described herein and/or to the Photo-sensitive MALDI Matrix Composite according to the present invention as described herein and/or to the use of a Photo-caged MALDI Matrix Compound according to the present invention as described herein apply mutatis mutandis to the method of identifying for a given type of analyte and predetermined vacuum conditions a suitable Photo-caged MALDI Matrix Compound according to the present invention as described herein, and vice versa.

In the method according to the present invention of identifying for a given type of analyte and predetermined vacuum conditions a suitable Photo-caged MALDI Matrix Compound, the Photoremovable Protecting Group Moiety B is a moiety of formula II as described above (or a moiety of formula II as described above as preferred).

A suitable uncaged MALDI matrix compound comprising the organic MALDI Matrix Compound Moiety A for use In the method according to the present invention of identifying a suitable Photo-caged MALDI Matrix Compound is derived from a corresponding compound which, upon common chemical modification, provides a heteroatom to which the Photoremovable Protecting Group Moiety B can be attached (as is explained above). Preferably, the organic MALDI Matrix Compound Moiety A comprised in the uncaged MALDI matrix compound is a moiety selected from the group consisting of a moiety of formula I-1, a moiety of formula I-2, a moiety of formula I-3, a moiety of formula I-4, a moiety of formula I-5 and a moiety of formula I-6. The said uncaged MALDI matrix compound is further suitable for the purpose of the present method if it can be combined with the given analyte to form a MALDI matrix composite and if a MALDI-MS analytical spectrum of the given analyte can be obtained with said MALDI matrix composite.

In the method according to the present invention of identifying for a given type of analyte and predetermined vacuum conditions a suitable Photo-caged MALDI Matrix Compound, assessing the dependency of MALDI analysis results preferably comprises, assessing the dependency of said MALDI analysis results from one or more parameters selected from (i) vacuum stability of each of the one or more derivatives of said suitable uncaged MALDI matrix compound, (ii) solubility of each of the one or more derivatives of said suitable uncaged MALDI matrix compound in one or more solvents suitable for performing a MALDI-MS or MALDI-MSI analysis of the given analyte, (iii) molar extinction coefficient of each of the one or more derivatives of said suitable uncaged MALDI matrix compound at a suitable wavelength, preferably at a wavelength in the range from 330-370 nm and (iv) the quality, preferably the resolution, of MALDI-MSI analytical spectra obtained under standardized conditions with the combinations of said type of analyte with both said suitable uncaged MALDI matrix compound and said one or more derivatives of said suitable uncaged MALDI matrix compound (wherein preferably the analyte is a standardized analyte based on a lipid ion “PI(38:4)” with m/z=885.6 as described by Fulop et al. in Anal. Chem. 85, 19 (2013) 9156-9163).

In the method according to the present invention of identifying for a given type of analyte and predetermined vacuum conditions a suitable Photo-caged MALDI Matrix Compound, identifying the suitable Photo-caged MALDI Matrix Compound preferably comprises a purposive expert selection from the assessed one or more derivatives of said suitable uncaged MALDI matrix compound by comparing the results of the said assessments (see above), based on the best results or the best combination of relevant results.

Under a further aspect, the present disclosure also comprises, and the present invention also includes—preferably as a variant of the Photo-sensitive MALDI Matrix Composite according to the present invention as described herein—a Photo-sensitive MALDI Matrix Composite (Variant) for use in a method of matrix-assisted laser desorption/ionization mass spectrometry, comprising or consisting of
C1) a matrix, completely or partially constituted (preferably completely constituted) by a Photo-caged MALDI Matrix Compound, comprising a Photoremovable Protecting Group Moiety B which is covalently bonded to an organic MALDI Matrix Compound Moiety A, and embedded in said matrix
C2) one or more than one analyte, to be analyzed in the method of matrix-assisted laser desorption/ionization mass spectrometry.

To the extent the Photo-sensitive MALDI Matrix Composite (Variant) described here above represents a variant of the Photo-sensitive MALDI Matrix Composite according to the present invention as described herein, all aspects of the present invention discussed herein in the context of the Photo-sensitive MALDI Matrix Composite according to the present invention as described herein apply mutatis mutandis to the Photo-sensitive MALDI Matrix Composite (Variant) described here above, and vice versa.

In the Photo-sensitive MALDI Matrix Composite (Variant) of said further aspect of the present disclosure as described above, the term “embedded” has the common meaning in the technical field, preferably as explained above.

In component C1) of the Photo-sensitive MALDI Matrix Composite (Variant) as described above, the matrix may be completely or partially constituted by a Photo-caged MALDI Matrix Compound as is described above with respect to the Photo-sensitive MALDI Matrix Composite according to the present invention.

Preferred is a Photo-sensitive MALDI Matrix Composite (Variant) according to the above-stated further aspect (or a Photo-sensitive MALDI Matrix Composite (Variant) according to the above-stated further aspect as described herein as preferred) wherein
the one or at least one of the more than one analyte is selected from the group consisting of proteins, peptides, lipids, nucleic acids, polysaccharides, glycopeptides, organometallic compounds and organic polymers, wherein preferably the molar mass of the analyte is in the range of from ≥100 Da to ≤200 000 Da, more preferably of from ≥100 Da to ≤50 000 Da and even more preferably of from ≥100 Da to ≤15 000 Da;
and/or
the total mass ratio of Photo-caged MALDI Matrix Compound (Variant): analyte is in the range of from ≥100:1 to ≤20 000:1, preferably of ≥500:1 to ≤10 000:1;
and/or
the Photo-sensitive MALDI Matrix Composite (Variant) is present in the solid state at 23° C. and 101.3 kPa;
and/or
the Photo-caged MALDI Matrix Compound is photo-cleavable, preferably photo-cleavable at the covalent bond (or at a covalent bond) between its moieties A and B, upon irradiation with radiation of a wavelength in the range of from ≥100 nm to ≤15 μm, preferably of from ≥150 nm to ≤12 μm, wherein preferably the radiation is or comprises radiation from a laser source, more preferably from a pulsed laser source and even more preferably from a modulated pulsed laser source.

The Photo-caged MALDI Matrix Compound present in the Photo-sensitive MALDI Matrix Composite (Variant) has a higher molecular mass and a lower vapor pressure (at 23° C.) than the corresponding MALDI matrix compound which can be released from it by photo-cleavage, as is likewise described herein with respect to the Photo-sensitive MALDI Matrix Composite according to the present invention.

The Photo-sensitive MALDI Matrix Composite (Variant) according to the above-stated further aspect as stated above may be present at 23° C. and ambient pressure (i.e. 101.3 kPa) in the liquid state, in particular in the form of an ionic liquid matrix (e.g. as described by Y. Yamazaki et al. in J Am Soc Mass Spectrom. 2020 Jun. 3; 31 (6): 1180-1188), or in the solid state. In a preferred variant of the present invention (and of all aspects of the present disclosure as described herein), the Photo-sensitive MALDI Matrix Composite is present in the solid state under the said conditions.

Similarly, the Photo-caged MALDI Matrix Compound as described herein (in particular the Photo-caged MALDI Matrix Compound according to the present invention as described herein) may be present at 23° C. and ambient pressure (i.e. 101.3 kPa) in the liquid state, in particular in the form of an ionic liquid (e.g. as described by Y. Yamazaki et al. in J Am Soc Mass Spectrom. 2020 Jun. 3; 31 (6): 1180-1188), see above, or in the solid state. In a preferred variant of the present invention, the Photo-caged MALDI Matrix Compound as described herein (in particularthe Photo-caged MALDI Matrix Compound according to the present invention as described herein) is present in the solid state under the said conditions.

Also preferred is a Photo-sensitive MALDI Matrix Composite (Variant) according to the above-stated further aspect (or a Photo-sensitive MALDI Matrix Composite (Variant) according to the above-stated further aspect as described herein as preferred), wherein the Photo-caged MALDI Matrix Compound present in the Photo-sensitive MALDI Matrix Composite (Variant) comprises
M1) an organic MALDI Matrix Compound Moiety A comprising
   two or more than two conjugated double bonds in its chemical structure,
   one or more than one heteroatom in its chemical structure, preferably selected from the group consisting of nitrogen, oxygen and sulfur, more preferably selected from the group consisting of nitrogen and oxygen,
   and preferably having
   a molar mass of ≥500 g/mol or less;
and
M2) a Photoremovable Protecting Group Moiety B,
   wherein the Photoremovable Protecting Group Moiety B is covalently bonded to the one or to one of the more than one heteroatoms of the organic MALDI Matrix Compound Moiety A,
   wherein preferably the covalent bond between the Photoremovable Protecting Group Moiety B and the one heteroatom, or between the Photoremovable Protecting Group Moiety B and one of the more than one heteroatoms, of the organic MALDI Matrix Compound Moiety A has a bond energy in the range of from ≥1 to ≤8 eV, preferably in the range of from 1.5 to ≤7 eV.

The above explanations regarding component C1) of the Photo-sensitive MALDI Matrix Composite according to the present invention, wherein the Photo-caged MALDI Matrix Compound is photo-cleavable upon irradiation with suitable radiation of a wavelength in the range of from ≥100 nm to ≤15 μm also apply mutatis mutandis to the Photo-sensitive MALDI Matrix Composite (Variant) according to the above-stated further aspect of the present disclosure.

As explained above, the organic MALDI Matrix Compound Moiety A (component C1) of the Photo-sensitive MALDI Matrix Composite (Variant)) preferably has a molar mass of ≥500 g/mol or less, more preferably in the range of from ≥100 g/mol to ≤500 g/mol, even more preferably of from ≥150 g/mol to ≤400 g/mol and yet even more preferably of from ≥150 g/mol to ≤350 g/mol.

The above explanations regarding suitable laser sources for providing the radiation for photo-cleaving the Photo-caged MALDI Matrix Compound according to the present invention as described herein also apply mutatis mutandis to the Photo-sensitive MALDI Matrix Composite (Variant) according to the above-stated further aspect of the present disclosure.

According to the present invention (and according to all aspects of the present disclosure as described herein), the organic MALDI Matrix Compound Moiety A comprises one or more than one heteroatom in its chemical structure. In the context of the present invention and disclosure, a “heteroatom” means an atom which is not a carbon atom and which preferably can be part of the structure of an organic molecule. Preferably, the heteroatom comprised by the chemical structure of the organic MALDI Matrix Compound Moiety A is selected from the group consisting of nitrogen, oxygen and sulfur, more preferably it is selected from the group consisting of nitrogen and oxygen.

Suitable MALDI matrix compounds, from which the MALDI Matrix Compound Moiety A (as defined above) can be derived, comprise MALDI matrix compounds known from the prior art, e.g. as disclosed by R. Zenobi et al. in “Mass Spectrometry Reviews” 17 (1998) 337-366, or by Q. Zhou et al. in Anal Bioanal Chem. (2021) 413:2599-2617, but preferably also comprise compounds suited for the very purpose which are still to be identified.

According to the present invention and according to all further aspects disclosed herein, a photoremovable protecting group is preferably attached to a MALDI matrix compound by (chemical modification) methods known in the art, to form a Photo-caged MALDI Matrix Compound, wherein the organic MALDI Matrix Compound MoietyA of said Photo-caged MALDI Matrix Compound is derived from said MALDI matrix compound and the Photoremovable Protecting Group Moiety B of said Photo-caged MALDI Matrix Compound is derived from said photoremovable protecting group. For example, the Photoremovable Protecting Group Moiety B of said Photo-caged MALDI Matrix Compound can in principle be derived from the photoremovable protecting groups known from the literature references cited here above.

Furthermore preferred is a Photo-sensitive MALDI Matrix Composite (Variant) according to the above-stated further aspect of the present disclosure (or a Photo-sensitive MALDI Matrix Composite (Variant) according to the above-stated further aspect as described herein as preferred), wherein the Photoremovable Protecting Group Moiety B of the Photo-caged MALDI Matrix Compound is selected from (or is derived from photoremovable protecting groups, respectively, as explained above, selected from) the group consisting of
arylcarbonylmethyl groups, preferably selected from the group consisting of phenacyl groups, o-alkylphenacyl groups, p-hydroxyphenacyl groups and benzoin groups;
nitroaryl groups, preferably nitrophenyl groups;
coumarin-4-ylmethyl groups;
arylmethyl groups;
a pivaloyl group;
carbonyl cyclo(hetero)alkyl groups;
carbonylaryl groups;
arylsulfonyl groups;
silyl groups;
2-hydroxycinnamyl groups;
α-keto amide groups;
α,β-unsaturated anilide groups;
methyl(phenyl)thiocarbamic acid groups;
thiochromone S,S-dioxide groups;
2-pyrrolidino-1,4-benzoquinone groups;
triazine groups;
arylmethyleneimino groups;
xanthene groups
and
pyronin groups,
or is derived from one of the aforementioned groups;
wherein preferably the Photoremovable Protecting Group Moiety B is selected from the group consisting of
   arylcarbonylmethyl groups, preferably selected from the group consisting of phenacyl groups, o-alkylphenacyl groups, p-hydroxyphenacyl groups and benzoin groups;
and
   nitroaryl groups, preferably nitrophenyl groups
or is derived from one of the aforementioned (preferred) groups.

In a more specific variant of said further aspect of the present disclosure (and of the present invention) is preferred a Photo-sensitive MALDI Matrix Composite (Variant) according to the above-stated further aspect (or a Photo-sensitive MALDI Matrix Composite (Variant) according to the above-stated further aspect as described herein as preferred) and a Photo-sensitive MALDI Matrix Composite according to the present invention as described herein (or a Photo-sensitive MALDI Matrix Composite according to the present invention as described herein as preferred), wherein the organic MALDI Matrix Compound Moiety A of the Photo-caged MALDI Matrix Compound is selected from the group consisting of
a moiety of formula I-1

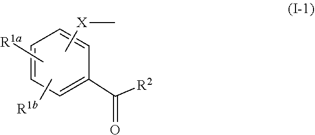

wherein
R^{1a} and R^{1b} are both independently of each other selected from the group consisting of hydrogen, hydroxy, carboxy, methyl and methoxy,
R² is selected from the group consisting of hydroxy and methyl and
X is selected from the group consisting of oxygen (—O—), —N(H)—, —N(CH₃)— and —N(C₂H₅)—;
a moiety of formula I-2

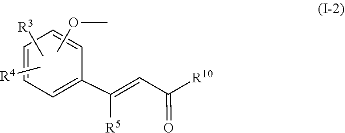

wherein
R³ and R⁴ are independently of each other selected from the group consisting of hydrogen, hydroxy, halogen and methoxy and
R⁵ is selected from the group consisting of hydroxy, methyl and cyano and
R¹⁰ is selected from the group consisting of hydroxy and methyl;
a moiety of formula I-3

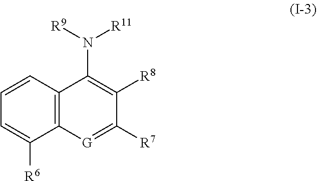

wherein
G is selected from the group consisting of nitrogen and C—H;
R⁶ is selected from the group consisting of hydrogen and amino,
R⁷ and R⁸ are independently of each other selected from the group consisting of hydrogen, amino and methyl, or together with the carbon atoms to which they are bonded, form a saturated or unsaturated five or six-membered ring,
R⁹ is selected from the group consisting of hydrogen, phenyl, saturated branched or unbranched alkyl having 1 to 4 carbon atoms and saturated branched or unbranched aminoalkyl having 1 to 4 carbon atoms and
R¹¹ is a bond or hydrogen
wherein preferably at least one of the groups G, R⁶, R⁷, R⁸, R⁹ or N—R¹¹ provides a heteroatom, preferably selected from oxygen and nitrogen, to which a Photoremovable Protecting Group Moiety B can be covalently bonded;
and
a moiety which is derived from a corresponding compound which, upon common chemical modification, provides a respective heteroatom, preferably selected from sulfur, oxygen and nitrogen, to which the Photoremovable Protecting Group Moiety B can be attached, wherein the corresponding compound is selected from the group consisting of 2-mercaptobenzothiazol, dithranol (also known as 1,8,9-trihydrox-yanthracene), 3-aminophthalhydrazide (also known as 3-APH), 2,3-dicyanohydroquinone (also known as DCH), 1,1′-binaphthyl-2,2′-diamine (also known as BNDM), 7-aminoquinoline, 2-(4-hydroxy-phenylazo)benzoic acid (also known as HABA), 6-aza-2-thiothymine (also known as ATT), p-nitroaniline (also known as PNA), 9-aminoacridine (also known as 9-AA), 2-(2-aminoethylamino)-5-nitropyridine, 1,8-bis(dimethylamino)naphthalene (also known as DMAN), 7-hydroxycoumarin-4-acetic acid, 7,8-dihydroxy-6-methoxycoumarin (also known as fraxetin), 6,7-dihydroxycoumarin, 7-hydroxycoumarin and 3-hydroxycoumarin (also known as 3-HC).

Where the organic MALDI Matrix Compound Moiety A of the Photo-caged MALDI Matrix Compound as described here above—for the Photo-sensitive MALDI Matrix Composite (Variant) according to the above-stated further aspect and for the Photo-sensitive MALDI Matrix Composite according to the present invention as described herein—is a moiety of formula I-1, this is preferably attached via the (free) bond of the group “X” to the Photoremovable Protecting Group Moiety B to form a Photo-caged MALDI Matrix Compound.

Where the organic MALDI Matrix Compound Moiety A of the Photo-caged MALDI Matrix Compound as described here above—for the Photo-sensitive MALDI Matrix Composite (Variant) according to the above-stated further aspect and for the Photo-sensitive MALDI Matrix Composite according to the present invention as described herein—is a moiety of formula I-2, this is preferably attached via the (free) bond of the oxygen atom attached to the phenyl ring to the Photoremovable Protecting Group Moiety B, to form a Photo-caged MALDI Matrix Compound.

Where the organic MALDI Matrix Compound Moiety A of the Photo-caged MALDI Matrix Compound as described here above—for the Photo-sensitive MALDI Matrix Composite (Variant) according to the above-stated further aspect of the present disclosure and for the Photo-sensitive MALDI Matrix Composite according to the present invention as described herein—is a moiety of formula I-3, this is preferably attached via a (free) bond of a nitrogen atom present in the moiety of formula I-3 to the Photoremovable Protecting Group Moiety B, to form a Photo-caged MALDI Matrix Compound.

Where the organic MALDI Matrix Compound Moiety A of the Photo-caged MALDI Matrix Compound as described here above, for the Photo-sensitive MALDI Matrix Composite (Variant) according to the above-stated further aspect and for the Photo-sensitive MALDI Matrix Composite according to the present invention as described herein, is a moiety of formula I-1, I-2 or I-3, preferred corresponding compounds from which such moieties 1-1, I-2 or I-3 are preferably derived are the same as is disclosed above in the context of the organic MALDI Matrix Compound Moiety A of the Photo-caged MALDI Matrix Compound according to the present invention In a further specific variant of the further aspect of the present disclosure and of the present invention is preferred a Photo-sensitive MALDI Matrix Composite (Variant) according to the above-stated further aspect (or a Photo-sensitive MALDI Matrix Composite (Variant) according to the above-stated further aspect as described herein as preferred) and a Photo-sensitive MALDI Matrix Composite according to the present invention as defined herein (or a Photo-sensitive MALDI Matrix Composite according to the present invention as described herein as preferred), wherein the Photoremovable Protecting Group Moiety B of the Photo-caged MALDI Matrix Compound is a nitrophenyl group, preferably defined by formula II

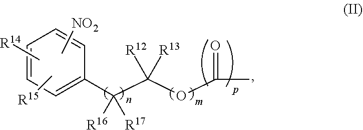

wherein
R¹² is selected from the group consisting of hydrogen and methyl;
R¹³ is selected from the group consisting of hydrogen, methyl and carboxy;
R¹⁴ is selected from the group consisting of hydrogen, methoxy, carboxy, nitro and —O(CH₂)_{I}COOH, wherein I is an integer in the range from 1 to 3;
R¹⁵ is selected from the group consisting of hydrogen and methoxy;
R¹⁶ and R¹⁷ are hydrogen or, together with the carbon atom to which they are bonded, form a carbonyl group; and
m, n and p are independently of each other 0 or 1;
and wherein preferably the MALDI Matrix Compound Moiety A is a moiety of formula I-1 as defined above.

A Photoremovable Protecting Group Moiety B of formula II as disclosed above with respect to the Photo-sensitive MALDI Matrix Composite (Variant) according to the above-stated further aspect of the present disclosure and with respect to the Photo-sensitive MALDI Matrix Composite according to the present invention as defined herein, is preferably attached to an organic MALDI Matrix Compound Moiety A via the (free) bond next to (and on the right side of) the letter “p” shown in formula II above.

A Photo-sensitive MALDI Matrix Composite according to the present invention as defined herein (or a Photo-sensitive MALDI Matrix Composite according to the present invention as described herein as preferred), or a Photo-sensitive MALDI Matrix Composite (Variant) according to the above-stated further aspect of the present disclosure is preferred in each case, wherein the Photo-caged MALDI Matrix Compound is selected from the group consisting of 1-(5-((4,5-dimethoxy-2-nitrobenzyl)oxy)-2-hydroxyphenyl)ethan-1-one, N¹-(4,5-dimethoxy-2-nitrobenzyl)naphthalene-1,5-diamine, 3-((4,5-dimethoxy-2-nitrobenzyl)oxy)-2H-chromen-2-one, 1-(2-((4,5-dimethoxy-2-nitrobenzyl)oxy)-6-hydroxyphenyl)ethan-1-one, 2-((4,5-dimethoxy-2-nitrobenzyl)thio)benzo[d]thiazole, 1-((4,5-dimethoxy-2-nitrobenzyl)oxy)-8-hydroxyanthracen-9(10H)-one, 4-((3-acetyl-4-hydroxyphenoxy)methyl)-3-nitrobenzoic acid, 4-(4-(1-(3-acetyl-4-hydroxyphenoxy)ethyl)-2-methoxy-5-nitrophenoxy)butanoic acid, 3-acetyl-4-hydroxyphenyl (4,5-dimethoxy-2-nitrobenzyl) carbonate and mixtures thereof. Accordingly, the Photo-caged MALDI Matrix Compound as listed here before represent preferred Photo-caged MALDI Matrix Compounds according to the present invention and according to the further aspect of the present disclosure stated above.

Under said further aspect as stated above, the present disclosure also comprises, and the present invention also includes—preferably as a variant of the use of a Photo-caged MALDI Matrix Compound according to the present invention as described herein—the use of a Photo-caged MALDI Matrix Compound of said further aspect (or the use of a Photo-caged MALDI Matrix Compound of said further aspect as described herein as preferred)
as matrix compound (and/or as MALDI matrix compound) in a method of matrix-assisted laser desorp-tion/ionization mass spectrometry
and/or
for preparing a Photo-sensitive MALDI Matrix Composite (Variant) according to the above-stated further aspect of the present disclosure (or a Photo-sensitive MALDI Matrix Composite (Variant) according to the above-stated further aspect of the present disclosure as described herein as preferred) or according to the present invention as defined herein (or a Photo-sensitive MALDI Matrix Composite according to the present invention as described herein as preferred).

To the extent the use of a Photo-caged MALDI Matrix Compound according to the said further aspect of the present disclosure as described herein represents a variant of the use of a Photo-caged MALDI Matrix Compound according to the present invention as described herein, all aspects of the present invention discussed herein in the context of the use of a Photo-caged MALDI Matrix Compound according to the present invention as described herein apply mutatis mutandis to the use of a Photo-caged MALDI Matrix Compound according to the said further aspect of the present disclosure as described herein, and vice versa.

Generally, all aspects discussed herein in the context of the Photo-sensitive MALDI Matrix Composite (Variant) according to the said further aspect of the present disclosure as described herein apply mutatis mutandis to the use of a Photo-caged MALDI Matrix Compound according to the said further aspect (i.e. said variant) of the present disclosure as described herein, and vice versa.

The use of the Photo-caged MALDI Matrix Compound according to the said further aspect of the present disclosure as described herein or according to the present invention as defined herein in each case comprises the use as matrix compound in a method of matrix-assisted laser desorption/ionization mass spectrometry where the Photo-caged MALDI Matrix Compound, without further modification as to its chemical structure, assumes the role of a MALDI matrix compound as is known in the art. Preferred is, however, in each case the use of the Photo-caged MALDI Matrix Compound according to the said further aspect of the present disclosure as described herein or according to the present invention as defined herein, wherein the Photo-caged MALDI Matrix Compound (present as matrix compound or MALDI matrix compound of a Photo-sensitive MALDI Matrix Composite (Variant) according to the said further aspect of the present disclosure as described herein or according to the present invention, as defined above) is first irradiated with a first radiation of a wavelength in the range of from ≥100 nm to ≤15 μm to release a MALDI matrix compound (as described in more detail below), and the so released MALDI matrix compound subsequently assumes the role of a MALDI matrix compound as is known in the art.

For the use of the Photo-caged MALDI Matrix Compound according to the said further aspect of the present disclosure as described herein or according to the present invention for preparing a Photo-sensitive MALDI Matrix Composite, the Photo-sensitive MALDI Matrix Composite can be prepared from a Photo-caged MALDI Matrix Compound, one or more than one analyte to be analyzed (as defined above) and optionally further components (e.g. including the MALDI matrix compound which can be obtained by photo-cleaving the Photo-caged MALDI Matrix Compound used) by methods known in the art.

More specifically is preferred the use of a Photo-caged MALDI Matrix Compound according to the said further aspect of the present disclosure as described herein or according to the present invention as described herein (or the use of a Photo-caged MALDI Matrix Compound according to the said further aspect of the present disclosure or according to the present invention, respectively, as described herein as preferred), wherein ≤20% of the total mass of a sample of the Photo-caged MALDI Matrix Compound evaporates after exposure of the sample to a vacuum of ≤3300 Pa, preferably of ≤10 Pa, for a time period in the range of from ≥36 hrs to ≤72 hrs, preferably of from ≥48 hrs to ≤96 hrs, more preferably of from ≥72 hrs to ≤120 hrs.

As explained above, stability of a MALDI matrix compound, viz. a Photo-caged MALDI Matrix Compound, against undesired premature evaporation is of particular value where a vacuum atmosphere needs to be applied for extended periods in MALDI-MS or MALDI-MSI methods.

Under said further aspect as stated above, the present disclosure further comprises, and the present invention also includes—preferably as a variant of the matrix-assisted laser desorption/ionization mass spectrometry method according to the present invention as described herein—a matrix-assisted laser desorp-tion/ionization mass spectrometry method of said further aspect (or a matrix-assisted laser desorption/ionization mass spectrometry method of said further aspect as described herein as preferred), comprising the following steps:
S1) providing or preparing a Photo-sensitive MALDI Matrix Composite (Variant) according to the above-stated further aspect of the present disclosure (or a Photo-sensitive MALDI Matrix Composite according to the above-stated further aspect of the present disclosure as described herein as preferred) or a Photo-sensitive MALDI Matrix Composite according to the present invention as described herein (or a Photo-sensitive MALDI Matrix Composite according to the present invention as described herein as preferred);
S2) irradiating the Photo-sensitive MALDI Matrix Composite as provided or prepared in step S1) with a first radiation of a wavelength suitable to release a MALDI matrix compound from the Photo-caged MALDI Matrix Compound present in the Photo-sensitive MALDI Matrix Composite, wherein the released MALDI matrix compound has a lower molecular mass and/or (preferably “and”) a higher vapor pressure at 25° C. than the Photo-caged MALDI Matrix Compound from which it was released, to receive a MALDI matrix composite, preferably comprising a MALDI matrix compound and, preferably embedded therein, one or more than one analyte to be analyzed;
S2a) irradiating the MALDI matrix composite received in step S2) with a second radiation of a nature and wavelength suitable to result in desorption/ionization of the one or more than one analyte from step S2), and preferably also suitable to result in desorption/ionization of the MALDI matrix compound received in step S2), and
S3) providing the one or more than one desorbed and/or ionized analyte from step S2a), and preferably the desorbed and/or ionized MALDI matrix compound from step S2a), to a mass spectrometer, preferably to the inlet of a mass spectrometer,
   wherein preferably at least the one or one of the more than one analyte is present in the gas phase.

To the extent the matrix-assisted laser desorption/ionization mass spectrometry method according to the said further aspect of the present disclosure as described herein represents a variant of the matrix-assisted laser desorption/ionization mass spectrometry method according to the present invention as described herein, all aspects of the present invention discussed herein in the context of the matrix-assisted laser desorption/ionization mass spectrometry method according to the present invention as described herein apply mutatis mutandis to the matrix-assisted laser desorption/ionization mass spectrometry method according to the said further aspect of the present disclosure as described herein, and vice versa.

Generally, all aspects discussed herein in the context of the Photo-sensitive MALDI Matrix Composite (Variant) according to the said further aspect of the present disclosure as described herein and/or to the use of a Photo-caged MALDI Matrix Compound according to the said further aspect of the present disclosure (i.e. of said variant) as described herein apply mutatis mutandis to the matrix-assisted laser desorption/ionization mass spectrometry method according to the said further aspect of the present disclosure as described herein (i.e. of said variant), and vice versa.

The Photo-sensitive MALDI Matrix Composite (Variant) in step S1) of the matrix-assisted laser desorp-tion/ionization mass spectrometry method according to the said further aspect of the present disclosure as described herein (as well as in step S1) of the matrix-assisted laser desorption/ionization mass spectrometry method according to the present invention as described herein) can preferably be prepared from a Photo-caged MALDI Matrix Compound (as described above), one or more than one analyte to be analyzed (as described above) and optionally further components (e.g. including the MALDI matrix compound which can be obtained by photo-cleaving the Photo-caged MALDI Matrix Compound used) by methods known in the art.

In step S2), of the matrix-assisted laser desorption/ionization mass spectrometry method according to the said further aspect of the present disclosure as described herein, irradiating the Photo-sensitive MALDI Matrix Composite with a first radiation of a suitable wavelength (as defined above or below) preferably has the effect that the Photo-caged MALDI Matrix Compound comprised by the Photo-sensitive MALDI Matrix Composite, which is likewise exposed to said first radiation, is photo-cleaved by the impact of the first radiation, preferably at the site of the covalent bonding between the Photoremovable Protecting Group Moiety B and the organic MALDI Matrix Compound Moiety A, so that a MALDI matrix compound is released. The MALDI matrix compound so released preferably corresponds to the compound from which the organic MALDI Matrix Compound Moiety A was derived.

Preferred is a matrix-assisted laser desorption/ionization mass spectrometry method according to the above-stated further aspect (or a matrix-assisted laser desorption/ionization mass spectrometry method according to the above-stated further aspect as described herein as preferred), or a matrix-assisted laser desorption/ionization mass spectrometry method according to the present invention as described herein (or a matrix-assisted laser desorption/ionization mass spectrometry method according to the present invention as described herein as preferred), wherein the wavelength of the first radiation applied in step S2) and/or the wavelength of the second radiation applied in step S2a) are independently of each other in the range of from ≥100 nm to ≤15 μm, preferably of from ≥250 nm to ≤10 μm,
wherein preferably
   the first radiation applied in step S2) is or comprises radiation from a laser source, preferably from a pulsed laser source, more preferably from a modulated pulsed laser source, wherein preferably the wavelength of the radiation is in the range of from ≥150 nm to ≤700 nm, more preferably of from ≥250 nm to ≤400 nm;
   and/or
   the second radiation applied in step S2a) is or comprises radiation from a laser source, preferably from a pulsed laser source, more preferably from a modulated pulsed laser source,
   wherein preferably the wavelength of the radiation is in the range of from ≥150 nm to ≤700 nm, preferably of from ≥250 nm to ≤400 nm;
   and/or
   the first radiation applied in step S2) and the second radiation applied in step S2a) both comprise radiation from a pulsed laser source, preferably from a modulated pulsed laser source,
   wherein preferably the wavelength of the radiation is in each case in the range of from ≥150 nm to ≤700 nm, preferably of from ≥250 nm to ≤400 nm.

In a preferred variant of the matrix-assisted laser desorption/ionization mass spectrometry method according to the above-stated further aspect as disclosed herein or according to the present invention as described herein, the first radiation applied in step S2) and/or the second radiation applied in step S2a) comprise in each case radiation from a pulsed laser source, preferably from a modulated pulsed laser source, wherein the wavelength of the radiation in each case comprises a wavelength selected from the group consisting of 266 nm, 337 nm, 355 nm, 2.94 μm and 10.6 μm, preferably selected from the group consisting of 266 nm, 337 nm and 355 nm.

Also preferred is a matrix-assisted laser desorption/ionization mass spectrometry method according to the above-stated further aspect (or a matrix-assisted laser desorption/ionization mass spectrometry method according to the above-stated further aspect as described herein as preferred), or a matrix-assisted laser desorption/ionization mass spectrometry method according to the present invention as described herein (or a matrix-assisted laser desorption/ionization mass spectrometry method according to the present invention as described herein as preferred), wherein the first radiation applied in step S2) and the second radiation applied in step S2a) are of equal nature and wavelength,
wherein preferably
   the irradiation in step S2) and the irradiation in step S2a) are performed with the same radiation,
   and/or
   the first radiation applied in step S2) and the second radiation applied in step S2a) are the same,
   and/or
   step S2) and step S2a) are performed in one single step.

In the preferred variant of the matrix-assisted laser desorption/ionization mass spectrometry method as described here above, release of the MALDI matrix compound from the Photo-caged MALDI Matrix Compound present in the Photo-sensitive MALDI Matrix Composite in step S2) and desorption/ionization of the analyte (and preferably the desorption/ionization of the MALDI matrix compound) in step S2a) are accomplished, preferably in one single step, by irradiating the Photo-sensitive MALDI Matrix Composite with radiation of a wavelength in a defined range (preferably with a wavelength in a preferred range as defined above) or with radiation of a preferred wavelength as defined above. In this preferred variant, the release of the MALDI matrix compound and the subsequent desorption/ionization of the analyte (and preferably the desorption/ionization of the MALDI matrix compound) both occur within an extremely short time interval. In this preferred variant, it is therefore not required to prepare and perform two different working steps which might otherwise require different machine settings and/or different sample preparation steps. This preferred variant of the matrix-assisted laser desorption/ionization mass spectrometry method as described here above is therefore a particularly beneficial and efficient variant of the method of the present invention.

Preferred is furthermore a matrix-assisted laser desorption/ionization mass spectrometry method according to the above-stated further aspect of the present disclosure (or a matrix-assisted laser desorption/ionization mass spectrometry method according to the above-stated further aspect as described herein as preferred), or a matrix-assisted laser desorption/ionization mass spectrometry method according to the present invention as described herein (or a matrix-assisted laser desorption/ionization mass spectrometry method according to the present invention as described herein as preferred), wherein step S2a) comprises irradiating the one or more than one desorbed but not yet ionized analyte, and preferably the desorbed MALDI matrix compound, with a third radiation from a laser source, preferably from a pulsed laser source, more preferably from a modulated pulsed laser source,
wherein preferably
   the wavelength of the third radiation is in the range of from ≥150 nm to ≤700 nm, preferably of from ≥250 nm to ≤400 nm
   and/or
   the third radiation is applied to the one or more than one desorbed but not yet ionized analyte when said analyte is in the gas phase, and preferably to the desorbed and/or ionized MALDI matrix compound when said MALDI matrix compound is in the gas phase.

In the preferred variant of the matrix-assisted laser desorption/ionization mass spectrometry method according to the above-stated further aspect of the present disclosure or according to the present invention as defined here above, the third radiation, which is combined with a common method of matrix-assisted laser desorption/ionization mass spectrometry, preferably serves for laser-induced postionization of the analyte and/or the MALDI matrix compound to enhance mass spectrometry imaging of certain classes of biomolecules. This method is known under the name “MALDI-2” and is e.g. described by J. Soltwisch et al. in SCIENCE, Vol. 348, Issue 6231 (2015), pp. 211-215, see above, or by J. Soltwisch et al. in Anal. Chem. 92, 13 (2020) pp. 8697-8703, see above.

Preferred is in addition a matrix-assisted laser desorption/ionization mass spectrometry method according to the above-stated further aspect of the present disclosure (or a matrix-assisted laser desorption/ionization mass spectrometry method according to the above-stated further aspect as described herein as preferred), or a matrix-assisted laser desorption/ionization mass spectrometry method according to the present invention as described herein (or a matrix-assisted laser desorption/ionization mass spectrometry method according to the present invention as described herein as preferred), wherein a vacuum is applied or maintained in steps S2), S2a) and/or S3) (preferably “and”), wherein preferably
the vacuum is applied or maintained for a time period of from ≥36 hrs to ≤72 hrs, preferably of from ≥48 hrs to ≤96 hrs, more preferably of from ≥72 hrs to ≤120 hrs,
and/or
the vacuum comprises a pressure of 3300 Pa or below, more preferably of 5 Pa or below and even more preferably of 0.1 Pa or below.

As explained above, stability of a MALDI matrix compound, viz. a Photo-caged MALDI Matrix Compound (according to both, the above-stated further aspect of the present disclosure or according to the present invention as defined here above) against undesired premature evaporation is of particular value where a vacuum atmosphere needs to be applied in MALDI-MS or MALDI-MSI methods. The above explanations regarding the higher molecular mass and the lower vapor pressure (at 23° C.) of a Photo-caged MALDI Matrix Compound as described herein and its lower vapor pressure (at 23° C.) compared to the MALDI matrix compound which can be released from it by photo-cleavage, stabilization of a MALDI matrix compound against evaporation and the making available of MALDI matrix compounds with a very low vapor pressure by converting them into corresponding Photo-caged MALDI Matrix Compounds do apply here mutatis mutandis.

Further preferred is a matrix-assisted laser desorption/ionization mass spectrometry method according to the above-stated further aspect of the present disclosure (or a matrix-assisted laser desorption/ionization mass spectrometry method according to the above-stated further aspect as described herein as preferred), or a matrix-assisted laser desorption/ionization mass spectrometry according to the present invention as described herein (or a matrix-assisted laser desorption/ionization mass spectrometry method according to the present invention as described herein as preferred), wherein step S1) comprises a step of co-crystallizing a Photo-caged MALDI Matrix Compound as defined herein (or a Photo-caged MALDI Matrix Compound as described herein as preferred), with the one or at least one of the more than one analyte as defined herein, preferably to form a Photo-sensitive MALDI Matrix Composite according to the present invention as described herein. Preferably, in this preferred variant of the matrix-assisted laser desorption/ionization mass spectrometry method according to the above-stated further aspect of the present disclosure or according to the present invention as defined herein the Photo-sensitive MALDI Matrix Composite and/or the Photo-caged MALDI Matrix Compound comprised by the Photo-sensitive MALDI Matrix Composite is in each case present in the solid state under the conditions applied in said method, more preferably herein the Photo-sensitive MALDI Matrix Composite and/or the Photo-caged MALDI Matrix Compound comprised by the Photo-sensitive MALDI Matrix Composite is in each case present in the solid state at 23° C. and 101.3 kPa.

Under said further aspect as stated above, the present disclosure furthermore comprises, and the present invention also includes, a Photo-caged MALDI Matrix Compound of said further aspect, as defined and described in the context of the Photo-sensitive MALDI Matrix Composite (Variant) of the further aspect of the present disclosure (or a Photo-caged MALDI Matrix Compound of said further aspect as described herein as preferred)—preferably as a variant of the Photo-caged MALDI Matrix Compound according to the present invention as described herein (or as a variant of the Photo-caged MALDI Matrix Compound according to the present invention as described herein as preferred).

To the extent the Photo-caged MALDI Matrix Compound according to the said further aspect of the present disclosure as described herein represents a variant of the Photo-caged MALDI Matrix Compound according to the present invention as described herein, all aspects of the present invention discussed herein in the context of the Photo-caged MALDI Matrix Compound according to the present invention as described herein apply mutatis mutandis to the Photo-caged MALDI Matrix Compound according to the said further aspect of the present disclosure as described herein, and vice versa.

Generally, all aspects discussed herein in the context of the Photo-sensitive MALDI Matrix Composite (Variant) according to the said further aspect of the present disclosure as described herein and/or of the use of a Photo-caged MALDI Matrix Compound according to the said further aspect of the present disclosure as described herein (i.e. of said variant) and/or of the matrix-assisted laser desorption/ionization mass spectrometry method according to the said further aspect of the present disclosure as described herein apply mutatis mutandis to the Photo-caged MALDI Matrix Compound according to the said further aspect of the present disclosure as described herein, and vice versa.

The present disclosure also comprises the following aspects A1 to A15 of the present invention, preferably according to the further aspect(s) of the present disclosure as described and explained in more detail here above:
A1. Photo-sensitive MALDI Matrix Composite (Variant) for use in a method of matrix-assisted laser de-sorption/ionization mass spectrometry, comprising or consisting of
   C1) a matrix, completely or partially constituted by a Photo-caged MALDI Matrix Compound, comprising a Photoremovable Protecting Group Moiety B which is covalently bonded to an organic MALDI Matrix Compound Moiety A
   and embedded in said matrix
   C2) one or more than one analyte, to be analyzed in the method of matrix-assisted laser desorp-tion/ionization mass spectrometry.
A2. Photo-sensitive MALDI Matrix Composite (Variant) according to aspect A1,
   wherein
      the one or at least one of the more than one analyte is selected from the group consisting of proteins, peptides, lipids, nucleic acids, polysaccharides, glycopeptides, organometallic compounds and organic polymers, wherein preferably the molar mass of the analyte is in the range of from ≥100 Da to ≤200 000 Da, more preferably of from ≥100 Da to ≤50 000 Da and even more preferably of from ≥100 Da to ≤15 000 Da;
   and/or
      the total mass ratio of Photo-caged MALDI Matrix Compound: analyte is in the range of from ≥100:1 to ≤20 000:1, preferably of ≥500:1 to ≤10 000:1;
   and/or
      the Photo-sensitive MALDI Matrix Composite is present in the solid state at 23° C. and 101.3 kPa;
   and/or
      the Photo-caged MALDI Matrix Compound is photo-cleavable, preferably photo-cleavable at the covalent bond between its moieties A and B, upon irradiation with radiation of a wavelength in the range of from ≥100 nm to ≤15 μm, preferably of from ≥150 nm to ≤12 μm, wherein preferably the radiation is or comprises radiation from a laser source, more preferably from a pulsed laser source and even more preferably from a modulated pulsed laser source.
A3. Photo-sensitive MALDI Matrix Composite (Variant) according to any of aspects A1 or A2, wherein the Photo-caged MALDI Matrix Compound comprises
   M1) an organic MALDI Matrix Compound Moiety A comprising
      two or more than two conjugated double bonds in its chemical structure,
      one or more than one heteroatom in its chemical structure
      and preferably having
      a molar mass of ≥500 g/mol or less;
   and
   M2) a Photoremovable Protecting Group Moiety B,
      wherein the Photoremovable Protecting Group Moiety B is covalently bonded to the one or to one of the more than one heteroatoms of the organic MALDI Matrix Compound Moiety A,
      wherein preferably the covalent bond between the Photoremovable Protecting Group Moiety B and the one heteroatom, or between the Photoremovable Protecting Group Moiety B and one of the more than one heteroatoms, of the organic MALDI Matrix Compound Moiety A has a bond energy in the range of from ≥1 to ≤8 eV, preferably in the range of from 1.5 to ≤7 eV.
A4. Photo-sensitive MALDI Matrix Composite (Variant) according to any of aspects A1 to A3, wherein the Photoremovable Protecting Group Moiety B of the Photo-caged MALDI Matrix Compound is selected from the group consisting of
   arylcarbonylmethyl groups, preferably selected from the group consisting of phenacyl groups, o-alkylphenacyl groups, p-hydroxyphenacyl groups and benzoin groups;
   nitroaryl groups, preferably nitrophenyl groups;
   coumarin-4-ylmethyl groups;
   arylmethyl groups;
   a pivaloyl group;
   carbonyl cyclo(hetero)alkyl groups;
   carbonylaryl groups;
   arylsulfonyl groups;
   silyl groups;
   2-hydroxycinnamyl groups;
   α-keto amide groups;
   α,β-unsaturated anilide groups;
   methyl(phenyl)thiocarbamic acid groups;
   thiochromone S,S-dioxide groups;
   2-pyrrolidino-1,4-benzoquinone groups;
   triazine groups;
   arylmethyleneimino groups;
   xanthene groups
   and
   pyronin groups,
   or is derived from one of the aforementioned groups;
   wherein preferably the Photoremovable Protecting Group Moiety B is selected from the group consisting of
      arylcarbonylmethyl groups, preferably selected from the group consisting of phenacyl groups, o-alkylphenacyl groups, p-hydroxyphenacyl groups and benzoin groups;
   and
      nitroaryl groups, preferably nitrophenyl groups.
A5. Photo-sensitive MALDI Matrix Composite (Variant) according to any of aspects A1 to A4, wherein the organic MALDI Matrix Compound Moiety A of the Photo-caged MALDI Matrix Compound is selected from the group consisting of
   a moiety of formula I-1

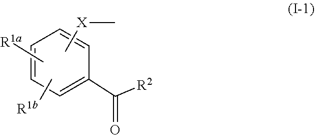

wherein
R^{1a} and R^{1b} are both independently of each other selected from the group consisting of hydrogen, hydroxy, carboxy, methyl and methoxy,
R² is selected from the group consisting of hydroxy and methyl and
X is selected from the group consisting of oxygen, —N(H)—, —N(CH₃)— and —N(C₂H₅)—;
a moiety of formula I-2

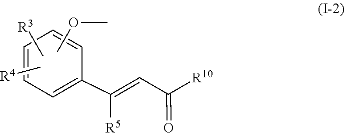

wherein
R³ and R⁴ are independently of each other selected from the group consisting of hydrogen, hydroxy, halogen and methoxy and
R⁵ is selected from the group consisting of hydroxy, methyl and cyano and
R¹⁰ is selected from the group consisting of hydroxy and methyl;
a moiety of formula I-3

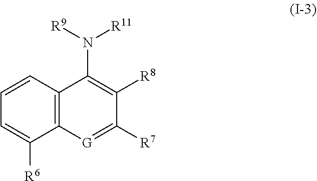

wherein
G is selected from the group consisting of nitrogen and C—H;
R⁶ is selected from the group consisting of hydrogen and amino,
R⁷ and R⁸ are independently of each other selected from the group consisting of hydrogen, amino and methyl, or together with the carbon atoms to which they are bonded, form a saturated or unsaturated five or six-membered ring,
R⁹ is selected from the group consisting of hydrogen, phenyl, saturated branched or unbranched alkyl having 1 to 4 carbon atoms and saturated branched or unbranched aminoalkyl having 1 to 4 carbon atoms and
R¹¹ is a bond or hydrogen,
wherein preferably at least one of the groups G, R⁶, R⁷, R⁸, R⁹ or N—R¹¹ provides a heteroatom, preferably selected from oxygen and nitrogen, to which a Photoremovable Protecting Group Moiety B can be covalently bonded;
and
a moiety which is derived from a corresponding compound which, upon common chemical modification, provides a respective heteroatom, preferably selected from sulfur, oxygen and nitrogen, to which the Photoremovable Protecting Group Moiety B can be attached, wherein the corresponding compound is selected from the group consisting of 2-mercaptobenzothiazol, dithranol, 3-aminophthalhydrazide, 2,3-dicyanohydroquinone, 1,1′-binaphthyl-2,2′-diamine, 7-aminoquinoline, 2-(4-hydroxy-phenylazo)benzoic acid, 6-aza-2-thiothymine, p-nitroaniline, 9-aminoacridine, 2-(2-aminoethylamino)-5-nitropyridine, 1,8-bis(dimethylamino)naphthalene, 7-hydroxycoumarin-4-acetic acid, 7,8-di-hydroxy-6-methoxycoumarin, 6,7-dihydroxycoumarin, 7-hydroxycoumarin and 3-hydroxycoumarin.
A6. Photo-sensitive MALDI Matrix Composite (Variant) according to any of aspects A1 to A5,
   wherein the Photoremovable Protecting Group Moiety B of the Photo-caged MALDI Matrix Compound is a nitrophenyl group, preferably defined by formula II

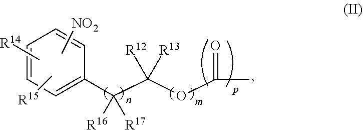

wherein
R¹² is selected from the group consisting of hydrogen and methyl;
R¹³ is selected from the group consisting of hydrogen, methyl and carboxy;
R¹⁴ is selected from the group consisting of hydrogen, methoxy, carboxy, nitro and —O(CH₂)_{I}COOH, wherein I is an integer in the range from 1 to 3;
R¹⁵ is selected from the group consisting of hydrogen and methoxy;
R¹⁶ and R¹⁷ are hydrogen or, together with the carbon atom to which they are bonded, form a carbonyl group; and
m, n and p are independently of each other 0 or 1;
and wherein preferably the MALDI Matrix Compound Moiety A is a moiety of formula I-1 as defined in aspect A5.
A7. Use of a Photo-caged MALDI Matrix Compound as defined in any of aspects A1 to A6
   as matrix compound in a method of matrix-assisted laser desorption/ionization mass spectrometry
   and/or
   for preparing a Photo-sensitive MALDI Matrix Composite (Variant) as defined in any of aspects 1 to 6.
A8. Use according to aspect A7, wherein ≤20% of the total mass of a sample of the Photo-caged MALDI Matrix Compound evaporates after exposure of the sample to a vacuum of ≤3300 Pa, preferably of ≤10 Pa, for a time period in the range of from ≥36 hrs to ≤72 hrs, preferably of from ≥48 hrs to ≤96 hrs, more preferably of from ≥72 hrs to ≤120 hrs.
A9. Matrix-assisted laser desorption/ionization mass spectrometry method, comprising the steps:
   S1) providing or preparing a Photo-sensitive MALDI Matrix Composite (Variant) as defined in any of aspects A1 to A6;
   S2) irradiating the Photo-sensitive MALDI Matrix Composite (Variant) as provided or prepared in step S1) with a first radiation of a wavelength suitable to release a MALDI matrix compound from the Photo-caged MALDI Matrix Compound present in the Photo-sensitive MALDI Matrix Composite,
      wherein the released MALDI matrix compound has a lower molecular mass and/or a higher vapor pressure at 25° C. than the Photo-caged MALDI Matrix Compound from which it was released,
      to receive a MALDI matrix composite, preferably comprising a MALDI matrix compound and, preferably embedded therein, one or more than one analyte to be analyzed;
   S2a) irradiating the MALDI matrix composite received in step S2) with a second radiation of a nature and wavelength suitable to result in desorption/ionization of the one or more than one analyte from step S2), and preferably also suitable to result in desorption/ionization of the MALDI matrix compound released in step S2),
   and
   S3) providing the one or more than one desorbed and/or ionized analyte from step S2a), and preferably the desorbed and/or ionized MALDI matrix compound from step S2a), to a mass spectrometer.
A10. Method according to aspect A9, wherein the wavelength of the first radiation applied in step S2) and/or the wavelength of the second radiation applied in step S2a) are independently of each other in the range of from ≥100 nm to ≤15 μm, preferably of from ≥250 nm to ≤10 μm,
   wherein preferably
      the first radiation applied in step S2) is or comprises radiation from a laser source, preferably from a pulsed laser source, more preferably from a modulated pulsed laser source,
      wherein preferably the wavelength of the radiation is in the range of from ≥150 nm to ≤700 nm, more preferably of from ≥250 nm to ≤400 nm;
      and/or
      the second radiation applied in step S2a) is or comprises radiation from a laser source, preferably from a pulsed laser source, more preferably from a modulated pulsed laser source,
      wherein preferably the wavelength of the radiation is in the range of from ≥150 nm to ≤700 nm, preferably of from ≥250 nm to ≤400 nm;
      and/or
      the first radiation applied in step S2) and the second radiation applied in step S2a) both comprise radiation from a pulsed laser source, preferably from a modulated pulsed laser source,
      wherein preferably the wavelength of the radiation is in each case in the range of from ≥150 nm to ≤700 nm, preferably of from ≥250 nm to ≤400 nm.
A11. Method according to any of aspects A9 to A10, wherein the first radiation applied in step S2) and the second radiation applied in step S2a) are of equal nature and wavelength,
   wherein preferably
      the irradiation in step S2) and the irradiation in step S2a) are performed with the same radiation,
      and/or
      the first radiation applied in step S2) and the second radiation applied in step S2a) are the same;
      and/or
      step S2) and step S2a) are performed in one single step.
A12. Method according to any of aspects A9 to A11, wherein step S2a) comprises irradiating the one or more than one desorbed but not yet ionized analyte, and preferably the desorbed and/or ionized MALDI matrix compound, with a third radiation from a laser source, preferably from a pulsed laser source, more preferably from a modulated pulsed laser source,
   wherein preferably
      the wavelength of the third radiation is in the range of from ≥150 nm to ≤700 nm, preferably of from ≥250 nm to ≤400 nm
      and/or
      the third radiation is applied to the one or more than one desorbed but not yet ionized analyte when said analyte is in the gas phase, and preferably to the desorbed and/or ionized MALDI matrix compound when said MALDI matrix compound is in the gas phase.
A13. Method according to any of aspects A9 to A12, wherein a vacuum is applied or maintained in steps S2), S2a) and/or S3), wherein preferably
   the vacuum is applied or maintained for a time period of from ≥36 hrs to ≤72 hrs, preferably of from ≥48 hrs to ≤96 hrs, more preferably of from ≥72 hrs to ≤120 hrs;
   and/or
   the vacuum comprises a pressure of 3300 Pa or below, more preferably of 5 Pa or below and even more preferably of 0.1 Pa or below.
A14. Method according to any of aspects A9 to A13, wherein step S1) comprises a step of co-crystallizing a Photo-caged MALDI Matrix Compound as defined in any of aspects A1 to A6 with the one or at least one of the more than one analyte as defined in any of aspects A1 to A2.
A15. Photo-caged MALDI Matrix Compound, as defined in any of aspects A1 to A6.

### FIGURES

The invention is further explained and illustrated by the appended figures, as explained here below:

FIG. 1: FIG. 1 shows on the left side crystals of the Photo-caged MALDI Matrix Compound of Ex. 1.1 formed by a spray method (cf. Example 3 below for details) on the surface of an ITO object slide analyzed by optical scanning in 20-fold magnification. FIG. 1 shows on the right side crystals of the Photo-caged MALDI Matrix Compound of Ex. 1.1 formed by a spray method (cf. Example 3 below for details) on the surface of a sliced sample of porcine brain analyzed by optical scanning in 20-fold magnification.

FIG. 2: FIG. 2 shows crystals of the Photo-caged MALDI Matrix Compound of Ex. 1.1 formed by a spray method (see above) on the surface of an ITO object slide analyzed by scanning electron microscopy in 5000-fold magnification.

FIG. 3: FIG. 3 shows crystals of the Photo-caged MALDI Matrix Compound of Ex. 1.1 formed by a spray method (see above) on the surface of an ITO object slide analyzed by scanning electron microscopy in 10000-fold magnification.

FIG. 4: FIG. 4 shows crystals of the Photo-caged MALDI Matrix Compound of Ex. 1.1 formed by a spray method (see above) on the surface of a sliced sample of porcine brain analyzed by scanning electron microscopy in 5000-fold magnification.

FIG. 5: FIG. 5 shows crystals of the Photo-caged MALDI Matrix Compound of Ex. 1.1 formed by a spray method (see above) on the surface of a sliced sample of porcine brain analyzed by scanning electron microscopy in 10000-fold magnification.

FIG. 6A: FIG. 6A shows a bright field scan of a sample of porcine brain (slice) on an object slide of glass, covered with Photo-caged MALDI Matrix Compound of Ex. 1.2 (as described below).

FIG. 6B to FIG. 6H: FIG. 6B to FIG. 6H show MALDI-MSI ion images of the sample of porcine brain from FIG. 6A at 50 μm spatial resolution in negative ion mode for different mass-to-charge ratios (FIG. 6B: m/z=726.6; FIG. 6C: m/z=728.6; FIG. 6D: m/z=750.6; FIG. 6E: m/z=766.6; FIG. 6F: m/z=788.6; FIG. 6G: m/z=885.6; FIG. 6H: m/z=888.7).

FIG. 7A: FIG. 7A shows a bright field scan of a sample of porcine brain (slice) on an object slide of glass, covered with Photo-caged MALDI Matrix Compound of Ex. 1.1 (as described below).

FIG. 7B to FIG. 7I: FIG. 7B to FIG. 7I show MALDI-MSI ion images of the sample of porcine brain from FIG. 7A at 50 μm spatial resolution in negative ion mode for different mass-to-charge ratios (FIG. 7B: m/z=726.6; FIG. 7C: m/z=728.6; FIG. 7D: m/z=750.6; FIG. 7E: m/z=766.6; FIG. 7F: m/z=788.6; FIG. 7G: m/z=885.6; FIG. 7H: m/z=888.7; FIG. 7I: m/z=904.7).

FIG. 8A: FIG. 8A shows a bright field scan of a sample of porcine brain (slice) on an object slide of glass, covered with Photo-caged MALDI Matrix Compound of Ex. 1.5 (as described below).

FIG. 8B to FIG. 8I: FIG. 8B to FIG. 8I show MALDI-MSI ion images of the sample of porcine brain from FIG. 8A at 50 μm spatial resolution in negative ion mode for different mass-to-charge ratios (FIG. 8B: m/z=726.6; FIG. 8C: m/z=728.6; FIG. 8D: m/z=750.6; FIG. 8E: m/z=766.6; FIG. 8F: m/z=788.6; FIG. 8G: m/z=885.6; FIG. 8H: m/z=888.7; FIG. 8I: m/z=904.7).

FIG. 9: FIG. 9 shows a MALDI-TOF MS spectrum of the Photo-caged MALDI Matrix Compound of Ex. 1.2 (DMNB-1,5-DAN) in negative ion mode.

FIG. 10: FIG. 10 shows a MALDI-TOF MS spectrum of the Photo-caged MALDI Matrix Compound of Ex. 1.1 (DMNB-2,5-DHAP) in negative ion mode.

FIG. 11: FIG. 11 shows a MALDI-TOF MS spectrum of the Photo-caged MALDI Matrix Compound of Ex. 1.5 (DMNB-2MBT) in negative ion mode.

FIG. 12: FIG. 12 shows optical images taken from ITO object slides carrying different conventional MALDI matrix compounds after the different incubation periods in a MALDI TOF mass spectrometer (cf. Example 4a).

FIG. 13: FIG. 13 shows optical images taken from ITO object slides carrying Photo-caged MALDI Matrix Compounds before and after incubation in a MALDI TOF mass spectrometer (cf. Example 4a).

FIG. 14: FIG. 14 shows the results of MALDI imaging measurements and of optical images made on samples of porcine brain tissue and on ITO slide surfaces before and after incubation under vacuum for 18 hours, using the Photo-caged MALDI Matrix Compound of Ex. 1.1 and (for comparison) the conventional MALDI matrix compound 2,5-DHAP (cf. Example 8).

FIG. 15: FIG. 15 shows the results of MALDI imaging measurements made on samples of porcine brain tissue on ITO slides before and after incubation under vacuum for 72 hours, using the Photo-caged MALDI Matrix Compound of Ex. 1.1 and (for comparison) the conventional MALDI matrix compound 2,5-DHAP (cf. Example 8).

FIG. 16: FIG. 16 shows a magnified view of a selected section (rectangular area overlapping sample sections C and D) from FIG. 15.

### EXAMPLES

The following examples are meant to further explain and illustrate the present invention without limiting its scope.

### Example 1: Synthesis of Photo-Caged MALDI Matrix Compounds According to the Invention

### Ex. 1.1: 1-(5-((4,5-Dimethoxy-2-nitrobenzyl)oxy)-2-hydroxyphenyl)ethan-1-one (DMNB-2,5-DHAP)

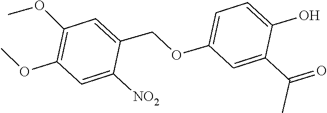 [SMILES: COc1cc(COc2ccc(O)c(C(C)=O)c2)c([N+](=O)[O-])cc1OC]

1-(Bromomethyl)-4,5-dimethoxy-2-nitrobenzene (2.5 g, 9.05 mmol, 1 eq), 1-(2,5 dihydroxy-phenyl)ethan-1-one (1.38 g, 9.05 mmol, 1 eq) and potassium carbonate (2.5 g, 18.11 mmol, 2 eq) were suspended in 50 ml of acetone. The mixture was stirred under reflux for 3 h after which it was filtered while hot. The filtrate was concentrated under reduced pressure to afford a crude brown solid which was purified via reversed-phase high performance liquid chromatography (RP-HPLC) to afford the desired product (see above, 1.06 g, 4.49 mmol) as a pale yellow solid in 50% yield. ¹H-NMR (600 MHz, Chloroform-d) δ [ppm] 11.94 (d, J=2.3 Hz, 1H), 7.78 (d, J=2.2 Hz, 1H), 7.35 (d, J=2.2 Hz, 1H), 7.32 (d, J=2.8 Hz, 1H), 7.23 (dt, J=9.2, 2.8 Hz, 1H), 6.97 (dd, J=8.9, 2.3 Hz, 1H), 5.47 (d, J=2.2 Hz, 2H), 4.00 (dd, J=9.9, 2.3 Hz, 6H), 2.64 (d, J=2.3 Hz, 3H). ¹¹C-NMR (151 MHz, Chloroform-d) δ 203.94, 157.41, 153.96, 150.20, 148.03, 139.18, 128.85, 125.23, 119.55, 119.35, 115.33, 109.48, 108.07, 68.28, 56.50, 56.43, 26.80. MS [M⁺Na]⁺ 370.14.

The Photo-caged MALDI Matrix Compound of Example 1.1 (Ex. 1.1) can be regarded as being prepared from the MALDI matrix compound 2,5-dihydroxyacetophenone (to form the MALDI Matrix Compound Moiety A) and the compound 1-(bromomethyl)-4,5-dimethoxy-2-nitrobenzene, serving to attach the photoremovable protecting group 4,5-dimethoxy2-nitrobenzyl to the MALDI matrix compound (to form the Photoremovable Protecting Group Moiety B). The Photoremovable Protecting Group Moiety B has the molecular formula C₉H₁₀O₄N (molar mass 196.1 g/mol) and is covalently bonded to an oxygen atom of the MALDI Matrix Compound Moiety A. The MALDI Matrix Compound Moiety A has the molecular formula C₈H₇O₃ and a molar mass of 151.1 g/mol.

### Ex. 1.2: N¹-(4,5-dimethoxy-2-nitrobenzyl)naphthalene-1,5-diamine (DMNB-1,5-DAN)

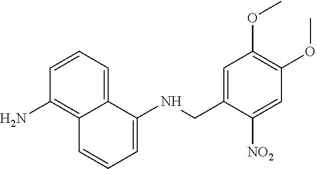 [SMILES: COc1cc(CNc2cccc3c(N)cccc23)c([N+](=O)[O-])cc1OC]

A mixture of 1-(bromomethyl)-4,5-dimethoxy-2-nitrobenzene (1 g, 3.62 mmol, 1 eq), naphthalene-1,5-diamine (1.15 g, 7.24 mmol, 2 eq) and cesium carbonate (1.18 g, 3.62 mmol, 1 eq) were suspended in 30 ml of DMF 30 ml. The mixture was stirred and heated to 60° C. for 5 h after which it was allowed to cool down to room temperature and it was concentrated to constant weight under reduced pressure. The solid residue was purified via RP-HPLC affording the desired product (see above, 0.22 g, 0.62 mmol) as a pale brown solid in 18% yield. ¹H-NMR (600 MHz, Chloroform-d) δ 7.78 (s, 1H), 7.37 (dt, J=8.6, 0.9 Hz, 1H), 7.32 (dd, J=8.5, 7.2 Hz, 1H), 7.28-7.19 (m, 2H), 7.16 (s, 1H), 6.83 (dd, J=7.2, 1.0 Hz, 1H), 6.45 (dd, J=7.0, 1.5 Hz, 1H), 5.32 (s, 2H), 4.95 (s, 2H), 3.97 (s, 3H), 3.73 (s, 3H). ¹³C-NMR (151 MHz, Chloroform-d) δ 153.72, 147.67, 142.99, 142.85, 140.18, 130.88, 125.48, 125.41, 124.24, 124.22, 110.84, 110.71, 110.40, 109.98, 108.47, 105.45, 56.40, 56.32, 46.56. MS [M⁺Na]⁺ 376.18.

### Ex. 1.3: 3-((4,5-Dimethoxy-2-nitrobenzyl)oxy)-2H-chromen-2-one (DMNB-3HC)

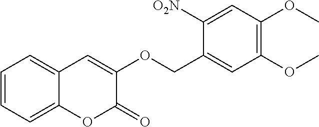 [SMILES: COc1cc(COc2cc3ccccc3oc2=O)c([N+](=O)[O-])cc1OC]

3-Hydroxy-2H-chromen-2-one (0.59 g, 3.62 mmol, 1 eq) and 1-(bromomethyl)-4,5-dimethoxy-2-nitrobenzene (1 g, 3.63 mmol, 1 eq) were dissolved in 50 ml of acetone followed by the addition of potassium carbonate (1.18 g, 3.63 mmol, 1 eq). The mixture was refluxed for 5 h after which volatiles were removed under reduced pressure and the residue was redissolved in dichloromethane and washed with 1M acq. NaOH solution (3×25 ml). The organic layer was dried over anhydrous Na₂S0₄ filtered and concentrated under reduced pressure to afford 1.2 g of a beige solid. The solid was purified via normal phase high performance liquid chromatography (NP-HPLC) affording the desired product (see above, 0,815 g, 2.28 mmol) as a beige solid in 63% yield. ¹H-NMR (600 MHz, Chloroform-d) δ 7.79 (s, 1H), 7.54 (s, 1H), 7.46 (dd, J=7.7, 1.5 Hz, 1H), 7.43 (ddd, J=8.7, 7.4, 1.6 Hz, 1H), 7.34 (dd, J=8.3, 1.1 Hz, 1H), 7.29 (td, J=7.5, 1.2 Hz, 1H), 7.05 (s, 1H), 5.71-5.39 (m, 2H), 4.02 (d, J=35.5 Hz, 6H). ¹³C-NMR (151 MHz, Chloroform-d) δ 157.36, 154.34, 149.79, 148.18, 143.03, 138.78, 128.97, 127.54, 126.78, 124.86, 119.35, 116.34, 115.26, 109.34, 107.96, 67.92, 56.67, 56.43. MS [M⁺Na]⁺ 380.32.

### Ex. 1.4: 1-(2-((4,5-Dimethoxy-2-nitrobenzyl)oxy)-6-hydroxyphenyl)ethan-1-one (DMNB-2,6-DHAP)

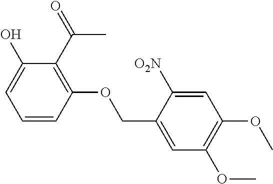 [SMILES: COc1cc(COc2cccc(O)c2C(C)=O)c([N+](=O)[O-])cc1OC]

1-(Bromomethyl)-4,5-dimethoxy-2-nitrobenzene (1 g, 3.62 mmol, 1 eq), 1-(2,6-dihydroxyphenyl)ethan-1-one (1.1 g, 7.24 mmol, 1 eq) and potassium carbonate (1 g, 7.24 mmol, 2 eq) were suspended in 25 ml of acetone. The mixture was stirred under reflux for 3 h after which it was filtered while hot. The filtrate was concentrated under reduced pressure to afford a crude brown solid which was purified via RP-HPLC to afford the desired product (see above, 0.51 g, 1.47 mmol) as a pale yellow solid in 40% yield. ¹H-NMR (600 MHz, Chloroform-d) δ 13.14 (s, 1H), 7.80 (s, 1H), 7.35 (t, J=8.3 Hz, 1H), 7.12 (s, 1H), 6.65 (dd, J=8.4, 1.0 Hz, 1H), 6.41 (dd, J=8.4, 1.0 Hz, 1H), 5.62 (s, 2H), 4.01 (s, 3H), 3.93 (s, 3H), 2.73 (s, 3H). ¹³C-NMR (151 MHz, Chloroform-d) δ 204.51, 164.67, 159.99, 153.79, 148.42, 139.69, 136.17, 127.38, 111.78, 111.64, 110.13, 108.36, 102.89, 68.56, 56.51, 56.48, 33.79.MS [M+Na]+370.33.

### Ex. 1.5: 2-((4,5-Dimethoxy-2-nitrobenzyl)thio)benzo[d]thiazole (DMNB-2MBT)

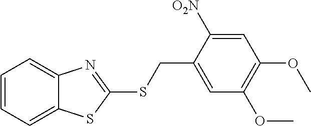 [SMILES: COc1cc(CSc2nc3ccccc3s2)c([N+](=O)[O-])cc1OC]

A mixture of benzo[d]thiazole-2(3H)-thione (0.48 g, 2.89 mmol, 0.8 eq), 1-(bromomethyl)-4,5-dimethoxy-2-nitrobenzene (1 g, 3.62 mmol, 1 eq) and triethylamine (0.76 ml, 5.43 mmol, 1.5 eq) were stirred in 30 ml of acetonitrile at room temperature for 18 h. Volatiles were removed under reduced pressure and the residue was purified via RP-HPLC affording the desired product (see above, 0.2 g 0.55 mmol) as a pale pink solid in 15% yield. ¹H-NMR (600 MHz, Chloroform-d) δ 7.95-7.80 (m, 1H), 7.76 (ddd, J=8.0, 1.2, 0.6 Hz, 1H), 7.72 (s, 1H), 7.49-7.38 (m, 2H), 7.32 (ddd, J=8.2, 7.3, 1.2 Hz, 1H), 4.98 (s, 2H), 3.95 (d, J=2.4 Hz, 6H). ¹³C-NMR (151 MHz, Chloroform-d) δ 166.43, 153.01, 152.85, 148.27, 140.23, 135.63, 128.44, 126.12, 124.38, 121.21, 121.05, 114.42, 108.32, 56.39, 56.38, 34.92. MS [M⁺Na]⁺ 385.05.

### Ex. 1.6: 1-((4,5-Dimethoxy-2-nitrobenzyl)oxy)-8-hydroxyanthracen-9(10H)-one (DMNB-Dithranol)

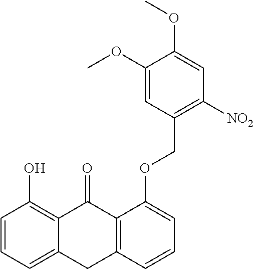 [SMILES: COc1cc(COc2cccc3c2C(=O)c2c(O)cccc2C3)c([N+](=O)[O-])cc1OC]

A mixture of 1,8-dihydroxyanthracen-9(10H)-one (1.64 g, 7.24 mmol, 1 eq), 1-(bromomethyl)-4,5-di-methoxy-2-nitrobenzene (1 g, 3.62 mmol, 1 eq) and potassium carbonate (1 g, 7.24 mmol, 2 eq) was refluxed in 50 ml of acetone for 4 h. Volatiles were removed and the residue was purified via RP-HPLC affording the desired product (see above, 0.25 g, 0.59 mmol) as a beige solid in 16% yield. ¹H NMR (600 MHz, Chloroform-d) δ 12.15 (s, 2H), 7.69 (s, 1H), 7.41 (dd, J=8.3, 7.5 Hz, 2H), 6.94 (dd, J=8.4, 1.0 Hz, 2H), 6.64 (dt, J=7.4, 0.9 Hz, 2H), 5.67 (s, 1H), 4.58 (t, J=7.1 Hz, 1H), 3.98 (s, 3H), 3.59 (s, 3H), 3.24 (d, J=7.2 Hz, 2H). ¹³C NMR (151 MHz, Chloroform-d) δ 193.32, 163.03, 152.07, 147.82, 145.52, 141.23, 136.14, 128.02, 119.81, 116.30, 115.36, 115.11, 108.07, 56.33, 56.17, 48.83, 44.77. MS [M⁺Na]⁺ 444.16.

Ex. 1.7: 4-((3-Acetyl-4-hydroxyphenoxy)methyl)-3-nitrobenzoic acid (CNB-2,5-DHAP)

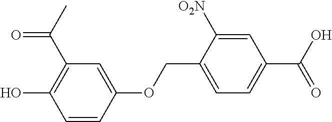 [SMILES: CC(=O)c1cc(OCc2ccc(C(=O)O)cc2[N+](=O)[O-])ccc1O]

### Ex. 1.7a: Methyl 4-(bromomethyl)-3-nitrobenzoate

4-(Bromomethyl)-3-nitrobenzoic acid (1 g, 3.65 mmol, 1 eq) was dissolved in 10 ml of methanol followed by the addition of concentrated sulfuric acid (400 μl). The mixture was refluxed for 2 h after which it was allowed to reach room temperature and it was concentrated under reduced pressure. The residue was purified via RP-HPLC to afford the desired product of Ex. 1.7a (0.95 g, 3.46 mmol) as a yellowish solid in 95% yield. ¹H-NMR (600 MHz, Chloroform-d) δ 8.68 (d, J=1.7 Hz, 1H), 8.26 (dd, J=8.0, 1.8 Hz, 1H), 7.74-7.64 (m, 1H), 4.86 (s, 2H), 4.00 (s, 3H), 3.99 (s, 1H), 3.54 (s, 1H). ¹³C-NMR (151 MHz, Chloroform-d) δ 164.45, 137.06, 134.14, 132.85, 131.72, 126.54, 125.83, 52.89, 27.95. MS [M⁺Na]⁺ 297.06.

### Ex. 1.7b: Methyl 4-((3-acetyl-4-hydroxyphenoxy)methyl)-3-nitrobenzoate

Methyl 4-(bromomethyl)-3-nitrobenzoate (0.95 g, 3.46 mmol, 1 eq), 1-(2,5 dihydroxyphenyl)ethan-1-one (0.53 g, 3.46 mmol, 1 eq) and potassium carbonate (0.96 g, 6.93 mmol, 2 eq) were suspended in 20 ml of acetone. The mixture was stirred under reflux for 3 h after which it was filtered while hot. The filtrate was concentrated under reduced pressure to afford a crude brown solid which was purified via RP-HPLC to afford the desired product of Ex. 1.7b (0.26 g, 0.75 mmol) as a pale yellow solid in 22% yield. ¹H-NMR (600 MHz, Chloroform-d) δ 11.93 (s, 1H), 8.82 (d, J=1.7 Hz, 1H), 8.37 (dd, J=8.2, 1.7 Hz, 1H), 8.05 (dt, J=8.1, 1.0 Hz, 1H), 7.33 (d, J=3.0 Hz, 1H), 7.22 (dd, J=9.0, 3.0 Hz, 1H), 6.98 (d, J=9.1 Hz, 1H), 5.73-5.30 (m, 2H), 4.02 (s, 3H), 2.65 (s, 3H). ¹³C-NMR (151 MHz, Chloroform-d) δ 203.90, 164.73, 157.53, 149.93, 146.88, 138.03, 134.47, 130.92, 128.88, 126.14, 124.88, 119.62, 119.38, 115.43, 67.82, 52.85, 26.81. MS [M⁺Na]⁺ 368.3.

### Ex. 1.7: See Above

Methyl 4-((3-acetyl-4-hydroxyphenoxy)methyl)-3-nitrobenzoate (0.26 g, 0.75 mmol, 1 eq) was dissolved in a 1:1 mixture of THE and water. Lithium hydroxide (0,036 g, 1.5 mmol, 2 eq) was added and the mixture was warmed up to 40° C. while stirring for 3 h. The mixture was allowed to cool down to room temperature after which trifuoroacetic acid was added until pH 3 followed by purification via RP-HPLC affording the desired product of Ex. 1.7 (see above, 0.15 g, 0.45 mmol) as a pale yellow solid in 60% yield. ¹H-NMR (600 MHz, DMSO-d6) δ 11.46 (s, 1H), 8.54 (d, J=1.7 Hz, 1H), 8.29 (dd, J=8.0, 1.8 Hz, 1H), 7.97 (d, J=8.0 Hz, 1H), 7.46 (t, J=2.6 Hz, 1H), 7.27 (dd, J=9.0, 3.1 Hz, 1H), 6.93 (d, J=9.1 Hz, 1H), 5.53 (s, 2H), 2.63 (s, 3H). ¹³C-NMR (151 MHz, DMSO) δ 203.99, 165.78, 155.81, 150.41, 147.67, 137.75, 134.56, 131.92, 129.99, 125.78, 124.76, 121.02, 119.13, 116.20, 67.66, 28.58. MS [M⁺Na]⁺ 354.06.

### Ex. 1.8: 4-(4-(1-(3-Acetyl-4-hydroxyphenoxy)ethyl)-2-methoxy-5-nitrophenoxy)butanoic acid (PMNB-2,5-DHAP)

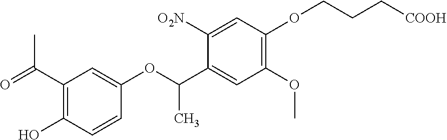 [SMILES: COc1cc(C(C)Oc2ccc(O)c(C(C)=O)c2)c([N+](=O)[O-])cc1OCCCC(=O)O]

### Ex. 1.8a: Methyl 4-(4-(1-hydroxyethyl)-2-methoxy-5-nitrophenoxy)butanoate

4-(4-(1-hydroxyethyl)-2-methoxy-5-nitrophenoxy)butanoic acid (0.6 g, 2 mmol, 1 eq) was dissolved in 5 ml of MeOH followed by the addition of trimethylsilyl chloride (505 μl, 4 mmol, 2 eq). The mixture was stirred at room temperature for 20 minutes. Volatiles were removed under reduced pressure affording the desired product of Ex. 1.8a (0.58 g, 1.84 mmol) as a white solid in 92% yield and sufficiently pure to be used in the next step. ¹H-NMR (600 MHz, Chloroform-d) δ 7.58 (s, 1H), 7.31 (s, 1H), 5.58 (q, J=6.3 Hz, 1H), 4.13 (tt, J=6.3, 3.1 Hz, 2H), 3.99 (s, 3H), 3.72 (s, 3H), 2.57 (t, J=7.2 Hz, 2H), 2.27-2.16 (m, 2H), 1.57 (d, J=6.3 Hz, 3H). ¹³C-NMR (151 MHz, Chloroform-d) δ 173.36, 154.13, 146.91, 139.55, 136.90, 109.09, 108.69, 68.23, 65.77, 56.34, 51.73, 30.37, 24.26. MS [M⁺Na]⁺ 336.29.

### Ex. 1.8b: Methyl 4-(4-(1-(3-acetyl-4-hydroxyphenoxy)ethyl)-2-methoxy-5-nitrophenoxy) butanoate

A mixture of 1-(2,5 dihydroxyphenyl)ethan-1-one (0.24 g, 1.55 mmol, 1 eq) and methyl 4-(4-(1-hydroxy-ethyl)-2-methoxy-5-nitrophenoxy)butanoate (0.51 g, 1.63 mmol, 1.05 eq) was dissolved in 1 ml of THE followed by the addition of diisopropyl azodicarboxylate (321 μl, 1.63 mmol, 1.05 eq) and triphenylphosphine (0.43 g, 1.63 mmol, 1.05 eq). The mixture was sonicated for 20 minutes after which the volatiles were removed under reduced pressure and the residue was purified via RP-HPLC affording the desired product of Ex. 1.8b (0.26 g, 0.58 mmol) as a pale brown solid in 37% of yield. ¹H-NMR (600 MHz, DMSO-d6) δ 12.17 (s, 1H), 11.38 (s, 1H), 7.59 (s, 1H), 7.28 (d, J=3.1 Hz, 1H), 7.22 (s, 1H), 7.13 (dd, J=9.0, 3.1 Hz, 1H), 6.84 (d, J=9.0 Hz, 1H), 5.94 (q, J=6.2 Hz, 1H), 4.06 (td, J=6.5, 2.4 Hz, 2H), 3.87 (s, 3H), 2.37 (t, J=7.3 Hz, 2H), 1.94 (h, J=6.6 Hz, 2H), 1.67 (d, J=6.2 Hz, 3H). ¹³C-NMR (151 MHz, Chloroform-d) δ 203.89, 173.30, 157.00, 154.47, 149.25, 147.34, 139.83, 134.03, 125.66, 119.44, 119.20, 115.38, 108.86, 108.47, 72.28, 68.19, 56.34, 51.73, 30.31, 26.66, 26.61, 24.22, 23.55. MS [M⁺Na]⁺ 470.14.

### Ex. 1.8: See Above

Methyl 4-(4-(1-(3-acetyl-4-hydroxyphenoxy)ethyl)-2-methoxy-5-nitrophenoxy)butanoate (0.27 g, 0.6 mmol, 1 eq) was dissolved in a 1:1 mixture of THE and water. Lithium hydroxide (0,029 g, 1.2 mmol, 2 eq) was added and the mixture was warmed up to 40° C. while stirring for 3 h. The mixture was allowed to cool down to room temperature after which trifuoroacetic acid was added until pH 3 followed by purification via RP-HPLC affording the desired product of Ex. 1.8 (see above, 0.22 g, 0.5 mmol) as a pale yellow solid in 85% yield. ¹H-NMR (600 MHz, DMSO-d6) δ 12.17 (s, 1H), 11.38 (s, 1H), 7.59 (s, 1H), 7.28 (d, J=3.1 Hz, 1H), 7.22 (s, 1H), 7.13 (dd, J=9.0, 3.1 Hz, 1H), 6.84 (d, J=9.0 Hz, 1H), 5.94 (q, J=6.2 Hz, 1H), 4.06 (td, J=6.5, 2.4 Hz, 2H), 3.87 (s, 3H), 2.37 (t, J=7.3 Hz, 2H), 1.94 (h, J=6.6 Hz, 2H), 1.67 (d, J=6.2 Hz, 3H). ¹³C-NMR (151 MHz, DMSO) δ 203.78, 174.43, 155.63, 154.06, 149.49, 147.35, 140.41, 133.01, 125.68, 120.87, 119.08, 117.44, 109.40, 109.01, 72.46, 68.35, 56.72, 30.39, 28.32, 24.44, 23.39. MS [M⁺Na]⁺ 456.10.

### Ex. 1.9: 3-Acetyl-4-hydroxyphenyl (4,5-dimethoxy-2-nitrobenzyl) carbonate (DMNB-2,5-DHAP)

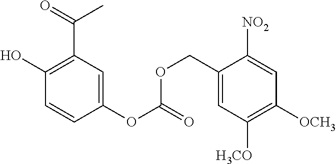 [SMILES: COc1cc(COC(=O)Oc2ccc(O)c(C(C)=O)c2)c([N+](=O)[O-])cc1OC]

1-(2,5-Dihydroxyphenyl)ethan-1-one (0.14 g, 0.91 mmol, 1 eq) was dissolved in 10 ml of dichloromethane (DCM) followed by the addition of triethylamine (0.25 ml, 1.82 mmol, 2 eq). The mixture was cooled down to 0° C. after which a solution of 4,5-dimethoxy-2-nitrobenzyl carbonochloridate (0.25 g, 0.91 mmol, 1 eq) in 10 ml of dichloromethane was dropped within 1 h. The mixture was stirred at 0° C. for one additional hour. The volatiles were removed and the solid residue was crystallized from MeOH/H₂O affording the desired product 10 (0,127 g, 0.32 mmol) as a pale yellow solid in 36% yield. ¹H-NMR (600 MHz, Chloroform-d) δ 12.18 (s, 1H), 7.79 (s, 1H), 7.60 (d, J=2.9 Hz, 1H), 7.34 (dd, J=9.0, 2.9 Hz, 1H), 7.14 (s, 1H), 7.03 (d, J=9.0 Hz, 1H), 5.71 (d, J=0.6 Hz, 2H), 4.02 (d, J=22.4 Hz, 6H), 2.65 (s, 3H). ¹¹C-NMR (151 MHz, Chloroform-d) δ 203.70, 160.33, 153.67, 153.41, 148.67, 142.43, 139.94, 129.47, 125.59, 122.19, 119.53, 119.23, 110.50, 108.35, 67.35, 56.58, 56.48, 26.73. MS [M⁺Na]⁺ 414.05.

### Example 2: Solubility Tests with Photo-Caged MALDI Matrix Compounds According to the Invention

Photo-caged MALDI Matrix Compounds according to the invention were prepared as described in Example 1 above and tested for solubility in different solvents (including solvent systems). The solvents (including solvent systems) used in this Example 2 are listed here below in Table 1:

TABLE 1
Solvents or solvent systems used for solubility tests

| Name | Solvent/solvent system |
|---|---|
| Solvent 1 | Acetonitrile |
| Solvent 2 | Acetonitrile/water (v/v 90/10) |
| Solvent 3 | Acetonitrile/water (v/v 90/10) + 0.1% trifluoroacetic acid |
| Solvent 4 | Acetonitrile/water (v/v 85/15) + 0.1% trifluoroacetic acid |
| Solvent 5 | Methanol/water (v/v 90/10) |
| Solvent 6 | Methanol/water (v/v 90/10) + 0.1% trifluoroacetic acid |
| Solvent 7 | Methanol/water (v/v 90/10) + 50 mM (final conc.) NH₄HCO₃ |
| Solvent 8 | Methanol/water (v/v 85/15) |
| Solvent 9 | Methanol/water (v/v 85/15) + 0.1% trifluoroacetic acid |
| Solvent 10 | Methanol/water (v/v 85/15) + 50 mM (final conc.) NH₄HCO₃ |

Around 1 mg of each Photo-caged MALDI Matrix Compound was weighed into an Eppendorf tube using an analytical balance. The calculated amount of selected solvent was added into the tube to generate a solution at a maximal concentration of 2.5 mg/mL. An ultrasonic bath was used for max. 3 min. to accelerate the dissolving process.

The solubilities found in the solubility tests of this Example 2 are shown in Table 2 below. The numbers of the compounds used in this Example 2 refer to the example numbers of Example 1 above. In Table 2 below, the mark “X” in a cell means, that the respective compound was completely dissolved in the respective solvent or solvent system in a concentration of 2.5 mg/mL. The mark “O” in a cell means, that the respective compound was not completely dissolved at an intended concentration of 2.5 mg/mL in the respective solvent or solvent system.

TABLE 2
Results of solubility tests with Photo-
caged MALDI Matrix Compounds

| Name | Ex. 1.1 | Ex. 1.2 | Ex. 1.3 | Ex. 1.4 | Ex. 1.5 | Ex 1.6 |
|---|---|---|---|---|---|---|
| Solvent 1 | X | X | X | X | X | ◯ |
| Solvent 2 | X | X | ◯ | X | X | ◯ |
| Solvent 3 | X | X | ◯ | X¹ | X¹ | ◯ |
| Solvent 4 | X¹ | X | ◯ | X | X | ◯ |
| Solvent 5 | ◯ | X | ◯ | ◯ | X | ◯ |
| Solvent 6 | X | X | ◯ | ◯ | ◯ | ◯ |
| Solvent 7 | ◯ | X | ◯ | ◯ | ◯ | ◯ |
| Solvent 8 | ◯ | X | ◯ | ◯ | ◯ | ◯ |
| Solvent 9 | ◯ | X¹ | ◯ | ◯ | ◯ | ◯ |
| Solvent 10 | ◯ | X | ◯ | ◯ | ◯ | ◯ |

¹Standard solvent (unless otherwise indicated)

From the results shown in Table 2 above it can be seen that the Photo-caged MALDI Matrix Compound according to the invention of Ex. 1.2 showed excellent solubility in the solvents or solvent systems under review and the Photo-caged MALDI Matrix Compounds according to the invention of Ex. 1.1, 1.4 and 1.5 showed very good solubility in the solvents or solvent systems under review.

### Example 3: Crystallisation Test with Photo-Caged MALDI Matrix Compound According to the Invention

The Photo-caged MALDI Matrix Compound of Ex. 1.1 according to the invention was prepared as described in Example 1 above and spray-tested for its crystallization properties in a spraying method on (i) an indium tin-oxide (ITO) object slide and (ii) on a sliced sample of porcine brain.

In each case, a solution of the Photo-caged MALDI Matrix Compound of Ex. 1.1 (concentration 2.5 mg/mL in acetonitrile/water v/v 4/1) was sprayed onto the surfaces of (i) an ITO object slide and (ii) on a sliced sample of porcine brain with a MALDI HTX M5 Sprayer™ (by HTX Technologies, LLC, USA).

The operating parameters applied were as follows: Temperature of spraying nozzle: 50° C./flow rate: 0.06 mL/min/speed of spraying nozzle: 1000 mm/min/spraying distance: 40 mm.

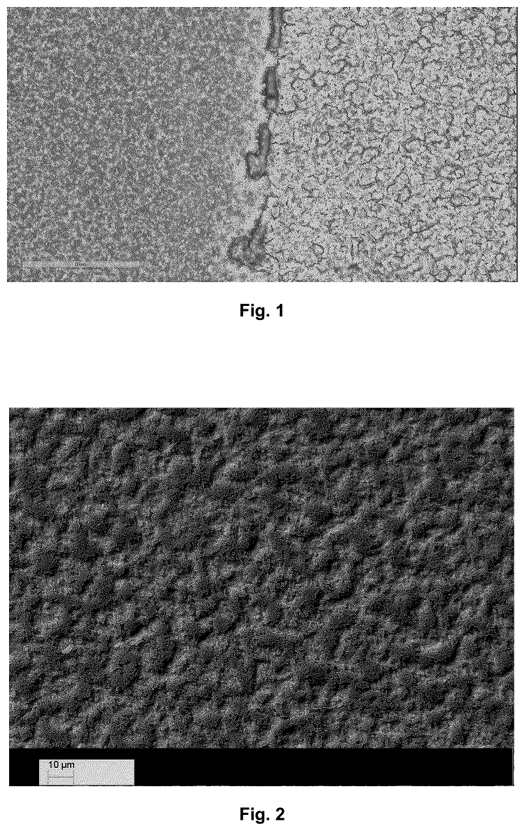

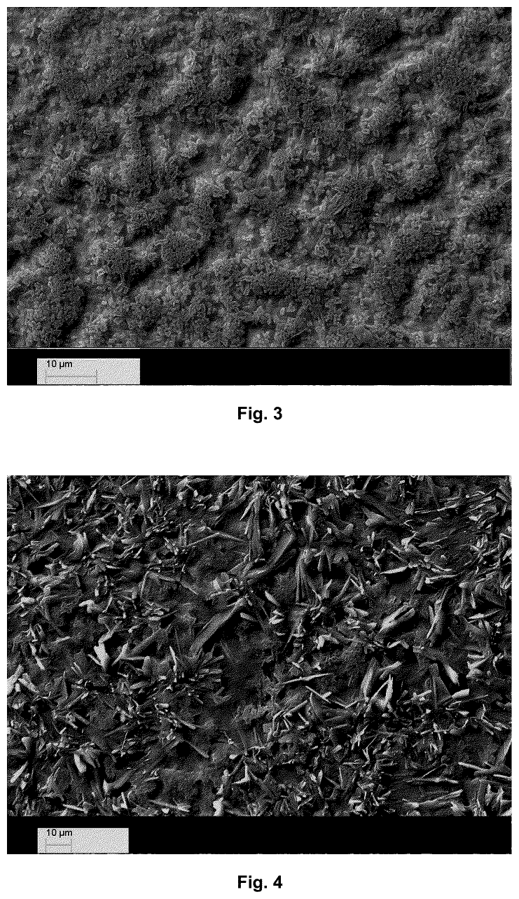

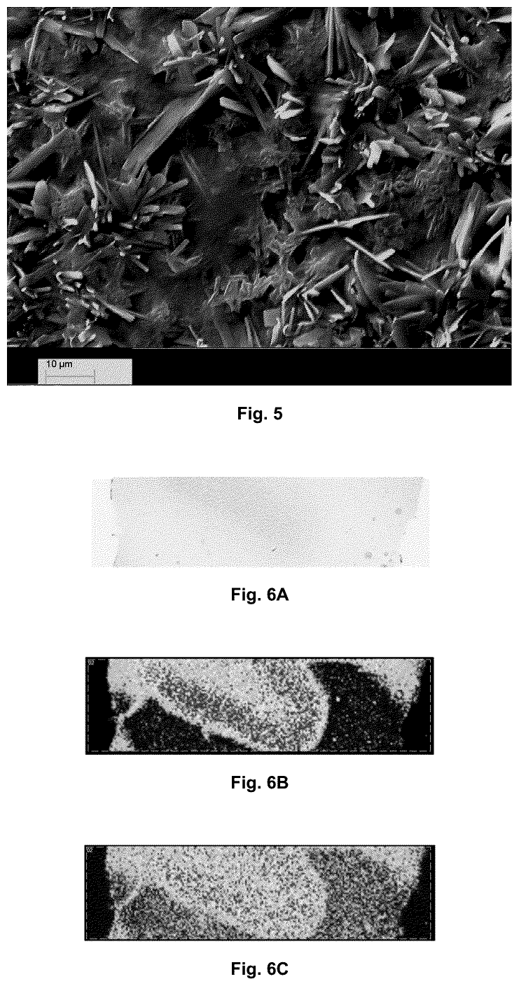

The resulting crystals on (i) an ITO object slide and (FIG. 1 to 3) (ii) on a sliced sample of porcine brain (FIG. 4 to 5) where then analyzed by optical scanning (in 20-fold magnification) and by scanning electron microscopy (in 5000-fold and 10000-fold magnification), as shown in FIG. 1 to 5.

It was found that the distribution of crystals of the Photo-caged MALDI Matrix Compound of Ex. 1.1 formed by this spray method was even in larger domains while in smaller domains further optimization would be required to improve evenness of the crystal distribution.

The average size of the resulting crystals of the Photo-caged MALDI Matrix Compound of Ex. 1.1 on the ITO object slide was about 1 μm (as determined by analysis of the scanning electron microscopy pho-tographies).

### Example 4: Test for Vacuum Stability of Photo-Caged MALDI Matrix Compound According to the Invention

For a determination of stability of the Photo-caged MALDI Matrix Compounds according to the present invention against evaporation upon exposure to a vacuum, the Photo-caged MALDI Matrix Compounds of Examples Ex. 1.1, Ex. 1.2, Ex. 1.3, Ex. 1.4, Ex. 1.5 and Ex. 1.6, respectively, were dissolved in acetonitrile (max. conc. 2.5 mg/mL in each case) and a drop of the resulting solutions (1 μL) was pipetted to an ITO object slide each.

Similarly, for comparison, solutions of the corresponding MALDI matrix compounds (2,5-dihydroxyacetophenone; 1,5-diaminonaphthalene; 3-hydroxycoumarin; 2,6-dihydroxyacetophenone, 2-mercaptobenzothiazole and dithranol, respectively) were dissolved in acetonitrile/water (v/v 1/1; conc. 5 mg/mL in each case) and a drop of the resulting solutions (1 μL) was pipetted to an ITO object slide each, too.

The six object slides carrying the Photo-caged MALDI Matrix Compounds and the six object slides carrying the corresponding MALDI matrix compounds were then transferred to a MALDI TOF spectrometer (Bruker UltrafleXtreme MALDI-TOF MS) and exposed to a vacuum (which was equivalent to usual operating conditions for a MALDI TOF process run) for a time period of 16 h.

Pictures were made by optical scanning (Aperio CS 2 scanner, Leica Biosystems, Wetzlar, Germany) of the ITO object slides of all 12 samples at the start of the experiment (t=0, before exposure to a vacuum, after evaporation of the solvent) and at the end of the experiment (t=16 h, after 16 hours exposure to a vacuum).

From a comparison of the pictures from optical scanning by visual inspection it was found that no visible losses of the six Photo-caged MALDI Matrix Compound samples could be identified, while considerable losses of all six corresponding MALDI matrix compounds were identified.

It can therefore be concluded that the Photo-caged MALDI Matrix Compounds showed a significantly improved stability against evaporation upon exposure to a vacuum when compared to their respective corresponding MALDI matrix compounds.

### Example 4a: Additional Test for Vacuum Stability of Photo-Caged MALDI Matrix Compound According to the Invention

A further test was made for determining the stability of Photo-caged MALDI Matrix Compounds according to the present invention against evaporation upon exposure to a vacuum.

Conventional MALDI matrix compounds 2,5-dihydroxybenzoic acid (2,5-DHB); 1,5-diaminonaphthalene (1,5-DAN); 3-hydroxycoumarin (3-HC); 2,6-dihydroxyacetophenone (2,5-DHAP); 2-mercaptobenzothiazole (2-MBT) and dithranol, respectively, were dissolved in acetonitrile/water (1:1 v/v) at a concentration of 5 mg/mL. For dithranol and 2-MBT, since not completely dissolved, the supernatant of each solution was used. 1 μL of each resulting solution was pipetted onto an ITO glass slide. After droplets had dried in the air, a first optical image (Aperio CS2 Scanner, Leica Biosystems, Wetzlar, Germany) of the whole ITO slides at 0 min. was recorded. The slides were then incubated at about 35° C. and a vacuum between about 3.5×10⁻⁶ mbar (t=0 min.) and about 1.4×10⁻⁷ mbar (t=892 min.) in an ultrafleXtreme™ MALDI TOF mass spectrometer (Bruker Daltonics) equipped with a Smartbeam-II Nd:YAG laser (355 nm) for time periods of 18 min., 80 min. and 892 min., respectively. After each time interval, additional optical images were taken of the slides. For 2,5-dihydroxyacetophenone (2,5-DHAP) a similar test was performed with a diluted solution (2.5 mg/mL).

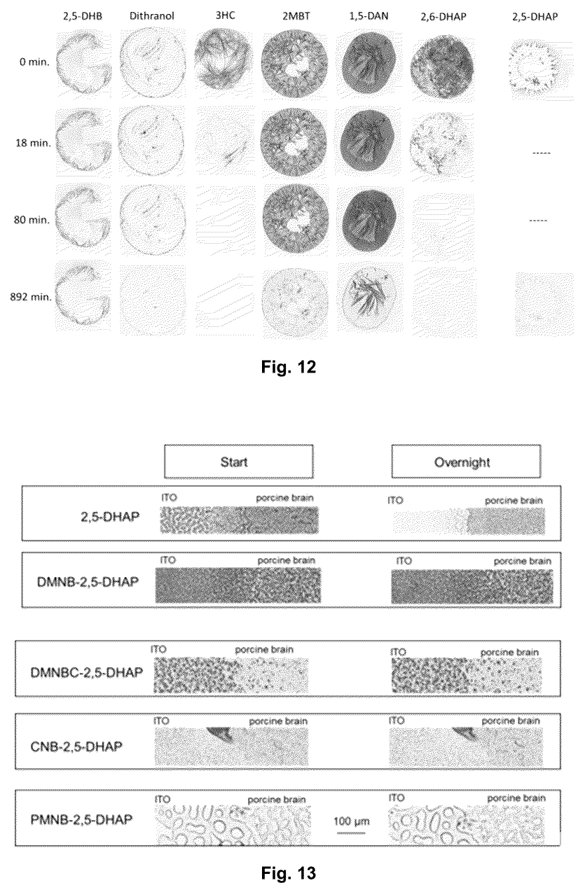

In FIG. 12, the optical images taken from the ITO object slides carrying the different MALDI matrix compounds after the different incubation periods are shown. It can be seen that the conventional MALDI matrix compounds evaporated over the time period(s) tested and the spots containing the MALDI matrix compounds gradually degraded, documenting the loss in (conventional) MALDI matrix compounds.

In a separate experiment, frozen porcine brain tissue was sectioned into 10 μm slices using a Leica CM1950 clinical cryostate (Leica Biosystems, Germany) at a chamber temperature of −15° C. and a head temperature of −10° C. The tissue sections were thaw-mounted onto ITO glass slides and dried for 20 min under vacuum. The ITO glass object slides with tissue sections were then sealed and stored at −80° C. until analysis. Shortly before usage, the slides were taken out from the freezer and thawed at room temperature for 20 min. under vacuum.

Photo-caged MALDI Matrix Compounds of Examples Ex. 1.1 (DMNB-2,5-DHAP), 1.7 (CNB-2,5-DHAP), 1.8 (PMNB-2,5-DHAP) and 1.9 (DMNBC-2,5 DHAP) were dissolved (Ex. 1.1: 2.5 mg/mL in standard solvent according to table 2 above; Ex.: 1.7 and 1.8:2.5 mg/mL in acetonitrile/water 7:3 (v/v)+0.1% trifluoroacetic acid; Ex. 1.9: saturated solution <2.5 mg/mL in acetonitrile/water 8:2 (v/v)+0.1% trifluoroacetic acid) and sprayed onto the ITO slides as prepared above, including to the porcine brain tissue sections thereof (see above) with a HTX M5 sprayer (HTX Technologies, USA), spray parameters: temperature of spray nozzle/tray: 50° C./25° C., Spray nozzle velocity 1000 mm/min; nozzle height: 40 mm. Twenty passes with a flow rate of 60 μL/min and a pressure of 10 psi were sprayed. Optical images of the sprayed slides were then recorded before incubation and after incubation in an ultrafleXtreme™ MALDI TOF mass spectrometer (see above) for 21 hrs at about 35° C. and a vacuum of up to about 1.4×10⁻⁷ mbar.

In FIG. 13, the optical images taken from the ITO object slides (glass and tissue sections) carrying the different Photo-caged MALDI Matrix Compounds according to the present invention before (“Start”) and after incubation for 21 hrs (“Overnight”) in the ultrafleXtreme™ MALDI TOF mass spectrometer are shown. It can be seen that the conventional MALDI matrix compound 2,5-DHAP was completely evaporated after incubation for 21 hrs, while the Photo-caged MALDI Matrix Compounds according to the present invention were still present on the porcine brain tissue sections of the ITO object slides with best results for Photo-caged MALDI Matrix Compounds of Examples Ex. 1.1 and Ex. 1.9.

### Example 5: Photo-Cleavage of Photo-Caged MALDI Matrix Compound According to the Invention and ReLease of Corresponding MALDI Matrix Compound

For a determination of the ability of the Photo-caged MALDI Matrix Compounds according to the present invention to be cleaved upon exposure to radiation of a wavelength in the range of from ≥100 nm to ≤15 μm, in particular upon exposure to radiation of a wavelength of 366 nm, the Photo-caged MALDI Matrix Compounds of Examples Ex. 1.1, Ex. 1.2, 1.4 and Ex. 1.5, respectively, were dissolved in acetonitrile (conc. 0.025 mg/mL in each case), filled into a glass cuvette and the UV absorption of the resulting solutions measured without and with exposure to UV radiation of a wavelength of 366 nm (HP G1103A 8453 UV-Vis Spectrophotometer).

Similarly, for comparison, solutions of the corresponding MALDI matrix compounds (2,5-dihydroxyacetophenone; 2,6-dihydroxyacetophenone and 2-mercaptobenzothiazole, respectively) were dissolved in acetonitrile (conc. 0.025 mg/mL in each case) and the UV absorption of the resulting solutions measured without and with exposure to UV radiation of a wavelength of 366 nm, too.

It was found that the UV absorption spectra of the MALDI matrix compounds 2,5-dihydroxyacetophenone, 2,6-dihydroxyacetophenone and 2-mercaptobenzothiazole did not change upon exposure to UV radiation of a wavelength of 366 nm, indicating that 2,5-dihydroxyacetophenone, 2,6-dihydroxyacetophenone and 2-mercaptobenzothiazole are stable towards UV irradiation at a wavelength of 366 nm.

For the above-stated Photo-caged MALDI Matrix Compounds it was found that their UV absorption maxima were changing upon exposure to UV radiation of a wavelength of 366 nm in a manner approximating the absorption maxima of the corresponding MALDI matrix compound in each case. It can therefore be concluded that the Photoremovable Protecting Group Moiety B (which is a nitrophenyl group of formula II as defined herein) is removed from the Photo-caged MALDI Matrix Compound in each case upon irradiation with UV radiation of a wavelength of 366 nm, to release a MALDI matrix compound corresponding to the respective organic MALDI Matrix Compound Moiety A of the Photo-caged MALDI Matrix Compound in each case.

A high absorption of a Photo-caged MALDI Matrix Compound in the UV wavelength range and the potential for its photo-cleavage in said UV wavelength range is beneficial because many commonly used MALDI TOF mass spectrometers use laser sources operating in this wavelength range (e.g. frequency-tripled Nd:YAG lasers, operating at a wavelength of 355 nm), so that said Photo-caged MALDI Matrix Compound can be photo-cleaved upon undergoing routine MALDI TOF mass spectrometer analysis.

### Example 6: MALDI-MSI Analytical Method Involving Photo-Sensitive MALDI Matrix Composites According to the Invention

### Example 6.1: MALDI-MSI of Porcine Brain with Photo-Caged MALDI Matrix Compound of Ex. 1.2

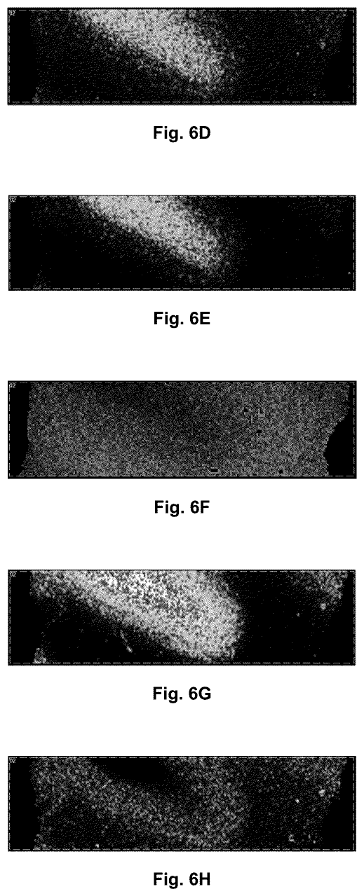

The Photo-caged MALDI Matrix Compound of Ex. 1.2 (DMNB-1,5-DAN) according to the invention was prepared as described in Example 1 above and sprayed onto a sample of sliced porcine brain (analyte; analogously to the method as described in Example 3 above) which was placed on a (glass) object slide. FIG. 6A shows a bright field scan of the porcine brain slice covered with Photo-caged MALDI Matrix Compound of Ex. 1.2.

The Photo-sensitive MALDI Matrix Composite according to the invention so received was then analyzed in a known MALDI mass spectrometry imaging (MALDI-MSI) method. Images received from this method show plots of ion intensity vs. relative position of the data from the sample.

Respective MALDI-MSI ion images at 50 μm spatial resolution in negative ion mode for different mass-to-charge ratios (“m/z-values”) are shown in FIGS. 6B to 6H. The bright areas in FIGS. 6B to 6H depict the presence of ions of specific m/z-values (FIG. 6B: m/z=726.6; FIG. 6C: m/z=728.6; FIG. 6D: m/z=750.6; FIG. 6E: m/z=766.6; FIG. 6F: m/z=788.6; FIG. 6G: m/z=885.6; FIG. 6H: m/z=888.7). Coloured information from the original MALDI-MSI ion images is not shown in the black-and-white format of FIGS. 6B to 6H. The mass-to-charge ratio pattern found by the MALDI-MSI method in the sample of sliced porcine brain is typical for a mixture of lipids in animal tissue.

### Example 6B: MALDI-MSI of Porcine Brain with Photo-Caged MALDI Matrix Compound of Ex. 1.1

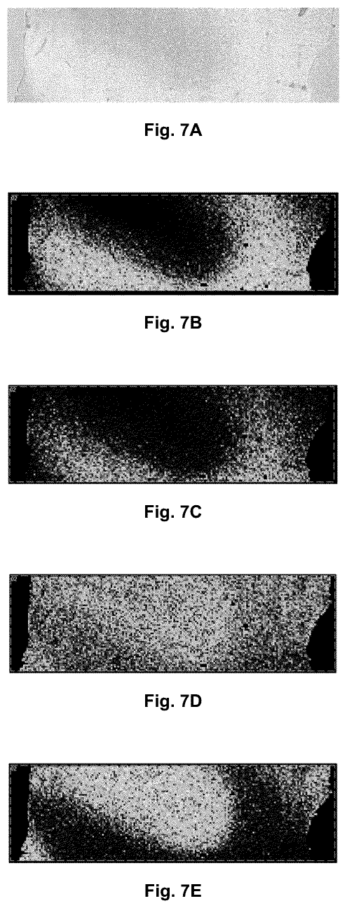

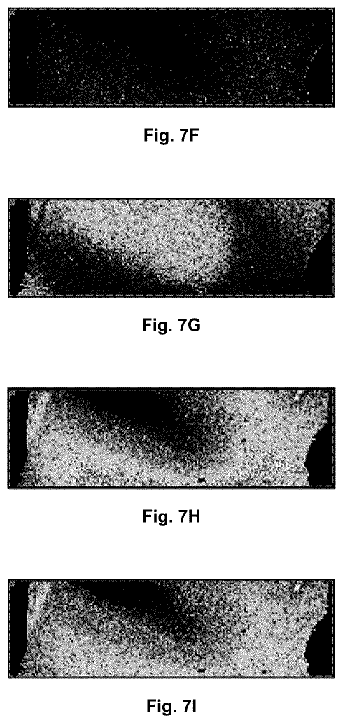

The Photo-caged MALDI Matrix Compound of Ex. 1.1 (DMNB-2,5-DHAP) according to the invention was prepared as described in Example 1 above and sprayed onto a sample of sliced porcine brain (analyte; analogously to the method as described in Example 3 above) which was placed on a (glass) object slide. FIG. 7A shows a bright field scan of the porcine brain slice covered with Photo-caged MALDI Matrix Compound of Ex. 1.1.

The Photo-sensitive MALDI Matrix Composite according to the invention so received was then analyzed in a known MALDI mass spectrometry imaging (MALDI-MSI) method as described above for Example 6.1.

Respective MALDI-MSI ion images at 50 μm spatial resolution in negative ion mode for different mass-to-charge ratios (“m/z-values”) are shown in FIGS. 7B to 7I. The bright areas in FIGS. 7B to 71 depict the presence of ions of specific m/z-values (FIG. 7B: m/z=726.6; FIG. 7C: m/z=728.6; FIG. 7D: m/z=750.6;

FIG. 7E: m/z=766.6; FIG. 7F: m/z=788.6; FIG. 7G: m/z=885.6; FIG. 7H: m/z=888.7; FIG. 7I: m/z=904.7). Coloured information from the original MALDI-MSI ion images is not shown in the black-and-white format of FIGS. 7B to 71. The mass-to-charge ratio pattern found by the MALDI-MSI method in the sample of sliced porcine brain is typical for a mixture of lipids in animal tissue.

### Example 6.3: MALDI-MSI of Porcine Brain with Photo-Caged MALDI Matrix Compound of Ex. 1.5

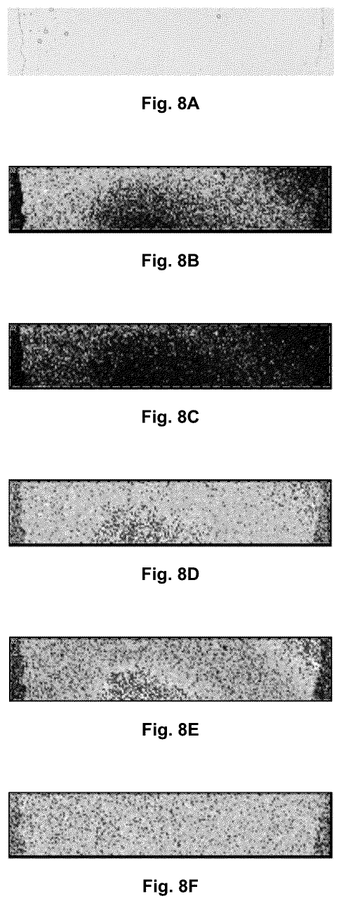

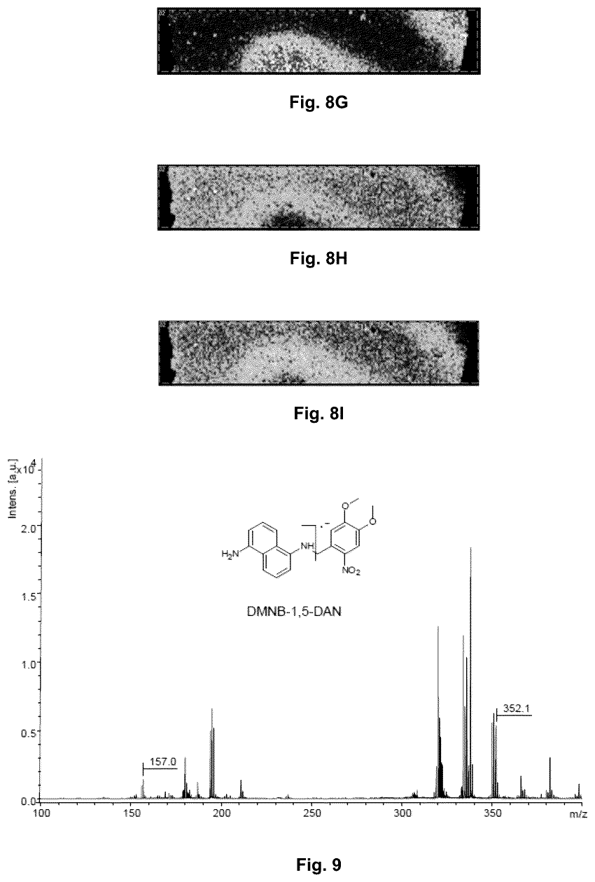

The Photo-caged MALDI Matrix Compound of Ex. 1.5 (DMNB-2,5-DHAP) according to the invention was prepared as described in Example 1 above and sprayed onto a sample of sliced porcine brain (analyte; analogously to the method as described in Example 3 above) which was placed on a (glass) object slide. FIG. 8A shows a bright field scan of the porcine brain slice covered with Photo-caged MALDI Matrix Compound of Ex. 1.5.

The Photo-sensitive MALDI Matrix Composite according to the invention so received was then analyzed in a known MALDI mass spectrometry imaging (MALDI-MSI) method as described above for Example 6.1.

Respective MALDI-MSI ion images at 50 μm spatial resolution in negative ion mode for different mass-to-charge ratios (“m/z-values”) are shown in FIGS. 8B to 8I. The bright areas in FIGS. 8B to 8I depict the presence of ions of specific m/z-values (FIG. 8B: m/z=726.6; FIG. 8C: m/z=728.6; FIG. 8D: m/z=750.6; FIG. 8E: m/z=766.6; FIG. 8F: m/z=788.6; FIG. 8G: m/z=885.6; FIG. 8H: m/z=888.7; FIG. 8I: m/z=904.7). Coloured information from the original MALDI-MSI ion images is not shown in the black-and-white format of FIGS. 8B to 8I. The mass-to-charge ratio pattern found by the MALDI-MSI method in the sample of sliced porcine brain is typical for a mixture of lipids in animal tissue.

From the results of this Example 6 it can be seen that the Photo-caged MALDI Matrix Compounds according to the present invention are suited for forming Photo-sensitive MALDI Matrix Composites according to the present invention. The Photo-caged MALDI Matrix Compounds according to the present invention and the Photo-sensitive MALDI Matrix Composites according to the present invention promoted desorption/ionization of various lipids from animal tissue.

It could also be shown in this Example 6 that the Photo-caged MALDI Matrix Compounds according to the present invention and the Photo-sensitive MALDI Matrix Composites according to the present invention enable mass spectrometry imaging (MSI) of lipids from animal tissue.

### Example 7: MALDI-TOF MS Spectra of Photo-Caged MALDI Matrix Compounds According to the Invention

### Example 7.1: Photo-Caged MALDI Matrix Compound of Ex. 1.2 (DMNB-1,5-DAN)

A MALDI-TOF MS spectrum of the Photo-caged MALDI Matrix Compound of Ex. 1.2 (DMNB-1,5-DAN) was recorded in negative ion mode (see FIG. 9). The fragments of the naphthalene-1,5-diamine radical ion (m/z=157.0) and the deprotonated molecule ion (m/z=352.1) could be detected.

### Example 7.2: Photo-Caged MALDI Matrix Compound of Ex. 1.1 (DMNB-2,5-DHAP)

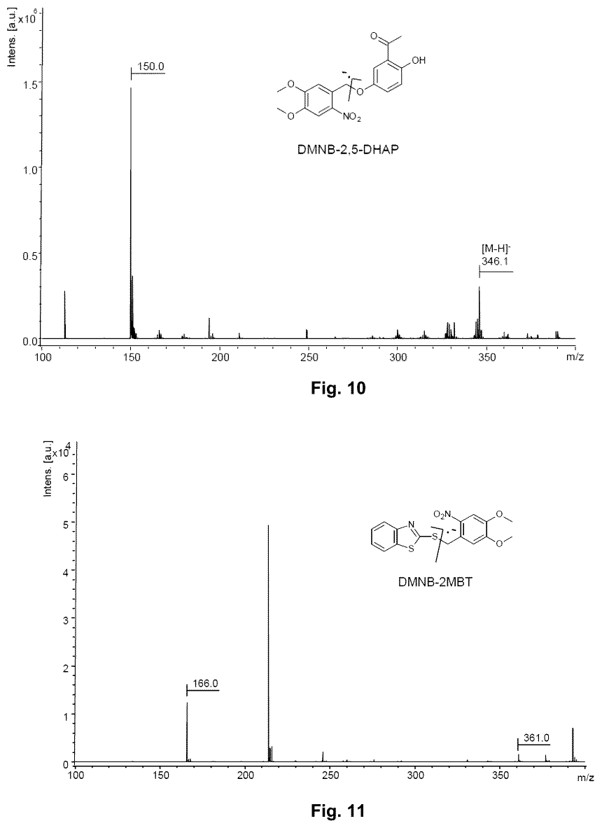

A MALDI-TOF MS spectrum of the Photo-caged MALDI Matrix Compound of Ex. 1.1 (DMNB-2,5-DHAP) was recorded in negative ion mode (see FIG. 10). The fragments of deprotonated 1-(2,5-dihydroxy-phenyl)ethan-1-one (m/z=150.0), 1-(2,5-dihydroxyphenyl) ethan-1-one as radical anion (m/z=151.0) and the deprotonated molecule ion (m/z=346.1) could be detected.

### Example 7.3: Photo-Caged MALDI Matrix Compound of Ex. 1.5 (DMNB-2MBT)

A MALDI-TOF MS spectrum of the Photo-caged MALDI Matrix Compound of Ex. 1.5 (DMNB-2MBT) was recorded in negative ion mode (see FIG. 11). The fragments of the benzo[d]thiazole-2(3H)-thione radical ion (m/z=166.0) and the deprotonated molecule ion (m/z=361.0) could be detected.

### Example 8: MALDI-TOF MSI Analyses of Lipids from Porcine Brain Tissue with Photo-Caged MALDI Matrix Compounds According to the Invention (Direct Tissue Analysis)

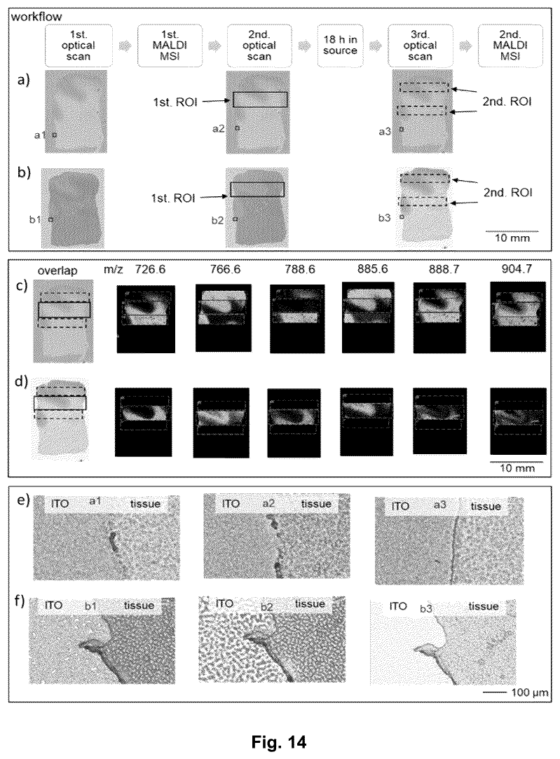

An ITO glass slide with two pieces of porcine brain tissue sections was prepared as described above (cf. Example 4a). An optical image was taken thereof for the registration process in the software FlexImaging. The workflow for the present example is explained below with reference to FIG. 14.

The Photo-caged MALDI Matrix Compound of Ex. 1.1 (DMNB-2,5-DHAP) and the conventional (uncaged) MALDI matrix compound 2,5-DHAP were both dissolved at concentrations of 2.5 mg/mL (acetonitrile/water 85:15, v/v+0.1% trifluoroacetic acid) and sprayed on top of the two different pieces of porcine brain tissue sections (preparation see above), using the spray parameters as described in Example 4a above. Homogenous and small crystals of DMNB-2,5-DHAP were subsequently observed on the ITO glass slide as well as on the porcine brain tissue section. 1^{st} and 2^{nd} MALDI imaging measurements and optical images were then made (optical images before and after 1^{st} MALDI imaging measurement and directly before 2^{nd} MALDI imaging measurement) of the Photo-caged MALDI Matrix Compound and of the conventional MALDI matrix compound on the ITO glass slides (glass surface sections and porcine tissue sections), respectively. After the 1^{st} MALDI imaging measurement, the slides were left inside the MALDI source for incubation (18 hrs, for incubation conditions see below) before the 2^{nd} MALDI imaging measurements were made.

MALDI imaging measurements were performed for the Photo-caged MALDI Matrix Compound of Ex. 1.1 and the conventional MALDI matrix compound 2,5-DHAP on a rapifleX™ MALDI TOF mass spectrometer (Bruker Daltonics) in reflector-negative ion mode in a mass range from m/z 100 to 1700 (ion suppression up to 80 m/z) using the FlexImaging 5.0 software (Bruker Daltonics). Inside the MALDI ion source, a vacuum of 2×10⁻⁷ mbar and a temperature of 35° C. (incubation conditions) were observed. The acquisition method was calibrated using a polyaniline solution and matrix. Settings: acquisition mode: 250 laser shots at 10 kHz repetition rate per position; spatial resolution: 50 μm; digitizer: 1.25 Gs/s; Ion Source: 1 to 20 kV; PIT: 2.47 kV; lens: 11.4 kV; Pulsed Ion Extraction: 110 ns. The laser energy was determined for each matrix. Data visualization was performed using FlexImaging 5.0 software (Bruker Daltonics). Total ion count (TIC) normalization was performed for all data.

In FIG. 14, the results of the MALDI imaging measurements and the optical images made are shown for the Photo-caged MALDI Matrix Compound of Ex. 1.1 (cf. under sections a), c) and e) of FIG. 14) and for the conventional MALDI matrix compound 2,5-DHAP (cf. under sections b), d) and f) of FIG. 14) Ion images (cf. under sections c) and d) of FIG. 14) of m/z 726.6, 766.6, 788.6, 885.6, 888.7, and 904.7 from the 1^{st} region of interest (“1^{st} ROI”, before incubation, see FIG. 14) and for the 2^{nd} region of interest (“2^{nd} ROI”, after incubation, see FIG. 14) on the ITO slides are both shown in FIG. 14 for ease of comparison. The regions marked “a1”, “a2”, “a3”, “b1”, “b2”, and “b3” in sections a) and b) of FIG. 14 are again displayed in 20-fold magnification in sections e) and f) of FIG. 14 (ion images from the 2^{nd} region of interest in section c) of FIG. 14 shown with 0-100% relative intensity scale for improved visibility, all other ion images shown with 0 to 60% relative intensity scale). Coloured information from the original MALDI-MSI ion images is not shown in the black-and-white format of FIG. 14.

Lipid identities were allocated according to Fulop et al. (2013), see above, as shown in table 3 below:

TABLE 3
Allocation of lipid identities according to Fülöp et al. (2013)

| m/z value | Lipid identity allocated |
|---|---|
| 726.6 | PE(p36:2) |
| 766.6 | PE(38:4) |
| 788.6 | PS(36:1) |
| 885.6 | PI(38:4) |
| 888.6 | SM4s(42:2) |
| 904.6 | SM4s(h42:2) |
| 1544.9 | GM1(38:1) |

From the results of the analyses of this Example 8 (documented in FIG. 14) it can be seen that ion images of lipids from porcine brain tissue made with the Photo-caged MALDI Matrix Compound of Ex. 1.1 (DMNB-2,5-DHAP) and such made with the conventional MALDI matrix compound 2,5-DHAP showed similar quality and resolution when made directly after preparation of the samples (cf. 1^{st} regions of interest in sections c) and d) of FIG. 14). However, when all samples were incubated in the MALDI source for 18 hrs under vacuum, ion images of the lipids from porcine brain tissue were still visible when DMNB-2,5-DHAP (according to the invention) was used as MALDI matrix compound, while no or nearly no ion images of the lipids from porcine brain tissue were visible when 2,5-DHAP (comparative example) was used as MALDI matrix compound.

Similar direct tissue MALDI TOF MSI analyses were also performed with the Photo-caged MALDI Matrix Compound of Ex. 1.2 vs. 1,5-DAN, with the Photo-caged MALDI Matrix Compound of Ex. 1.4 (2,6-DHAP known to be extremely sensitive to vacuum conditions and volatile) and with the Photo-caged MALDI Matrix Compound of Ex. 1.5 vs. 2-MBT with similar results, i.e. that images of the lipids from porcine brain tissue were still visible when the Photo-caged MALDI Matrix Compounds according to the present invention were used as MALDI matrix compounds, while intensities of ion images of the lipids from porcine brain tissue decreased significantly where the corresponding conventional (uncaged) MALDI matrix compounds were used (in comparative examples).

Best results were achieved in the analyses of this Example 8 with the Photo-caged MALDI Matrix Compound of Ex. 1.1 (DMNB-2,5-DHAP).

### Example 9: MALDI-TOF MSI Analyses of Lipids from Porcine Brain Tissue with Photo-Caged MALDI Matrix

Compound according to the invention (direct tissue analysis)—extended vacuum conditions A similar direct tissue MALDI TOF MSI analysis as described in Example 8 above was performed with the Photo-caged MALDI Matrix Compound of Ex. 1.1 (DMNB-2,5-DHAP) and the conventional (uncaged) MALDI matrix compound 2,5-DHAP (comparative example) on tissue sections of porcine brain, mounted on ITO slides.

MALDI imaging measurements were made (about 45 min. per measurement) of the Photo-caged MALDI Matrix Compound and of the conventional MALDI matrix compound on the porcine brain tissue sections, respectively, directly after preparation of the samples (“Start”) and (deviating from the protocol described in Example 8 above) after 72 hours of incubation in the MALDI source under vacuum.

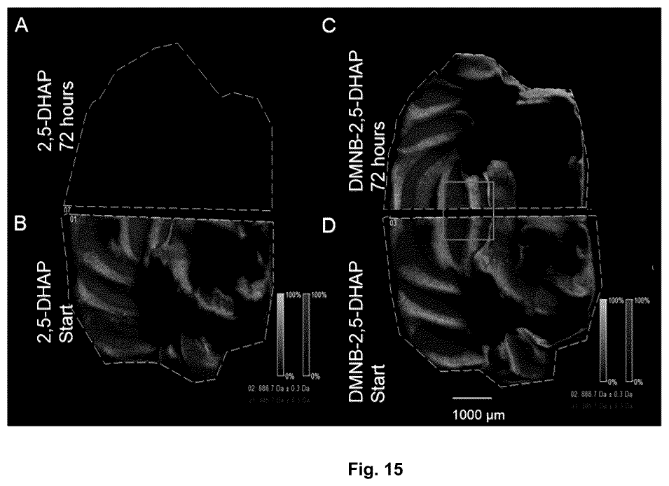

The result of this Example 9 is shown in FIG. 15. It can be seen from FIG. 15 that ion images of two typical lipids (m/z=888.7 and m/z=885.7) from the porcine brain tissue samples made with the Photo-caged MALDI Matrix Compound of Ex. 1.1 (DMNB-2,5-DHAP) and such made with the conventional MALDI matrix compound 2,5-DHAP showed similar quality and resolution when made directly after preparation of the samples (cf. pictures of porcine brain tissue samples marked “B” for 2-5-DHAP and “D” for DMNB-2,5-DHAP). However, when the samples were incubated in the MALDI source for 72 hrs under vacuum, ion images of the said lipids from porcine brain tissue were still visible (and were nearly unchanged) when DMNB-2,5-DHAP (according to the invention) was used as MALDI matrix compound, while no more ion images of the said lipids from porcine brain tissue were visible after incubation when 2,5-DHAP (comparative example) was used as MALDI matrix compound (cf. pictures in FIG. 15 of porcine brain tissue samples marked “A” for 2-5-DHAP and “C” for DMNB-2,5-DHAP).

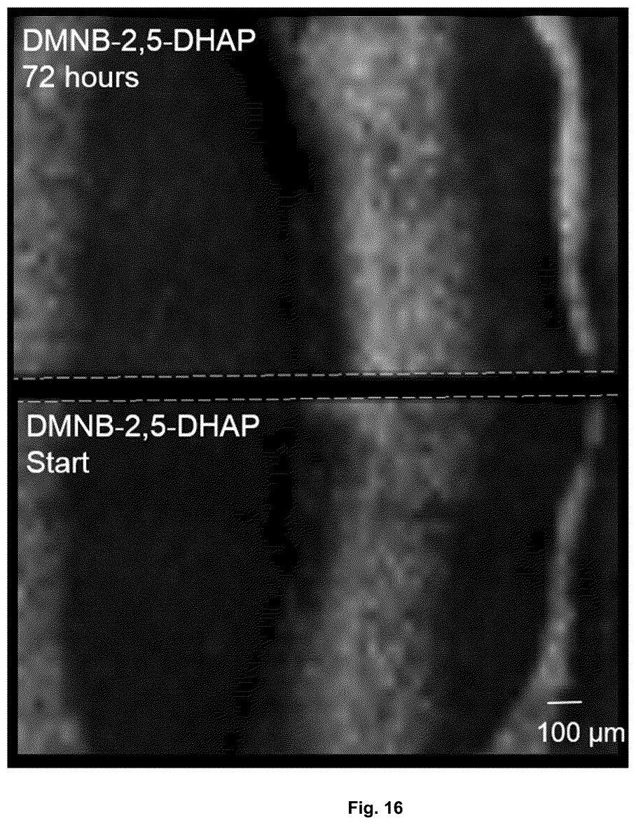

FIG. 16 shows a magnified portion of FIG. 15 (marked as rectangular section in FIG. 15), for better visibility and comparison of spectrum quality and resolution.

Coloured information from the original MALDI-MSI ion images is not shown in the black-and-white format of FIGS. 15 and 16.

We claim:

1. Photo-caged MALDI Matrix Compound for use in a method of matrix-assisted laser desorption/ionization mass spectrometry, comprising or consisting of:
a Photoremovable Protecting Group Moiety B which is covalently bonded to an organic MALDI Matrix Compound Moiety A,
wherein
the organic MALDI Matrix Compound Moiety A is a moiety selected from the group consisting of
a moiety of formula I-1 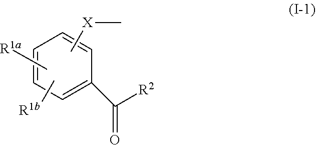
wherein
R^{1a} and R^{1b} are both independently of each other selected from the group consisting of hydrogen, hydroxy, carboxy, methyl and methoxy,
R² is selected from the group consisting of hydroxy and methyl and
X is selected from the group consisting of oxygen, —N(H)—, —N(CH₃)— and —N(C₂H₅)—,
wherein the moiety of formula I-1 is attached to the Photoremovable Protecting Group Moiety B via the free bond to the right of group X;
a moiety of formula I-2 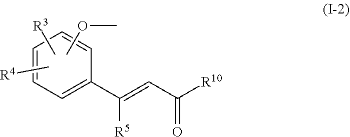
wherein
R³ and R⁴ are independently of each other selected from the group consisting of hydrogen, hydroxy, halogen and methoxy and
R⁵ is selected from the group consisting of hydroxy, methyl and cyano and
R¹⁰ is selected from the group consisting of hydroxy and methyl,
wherein the moiety of formula I-2 is attached to the Photoremovable Protecting Group Moiety B via the free bond to the right of the oxygen atom attached to the phenyl ring;
a moiety of formula I-3 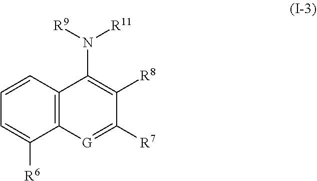
wherein
G is selected from the group consisting of nitrogen and C—H;
R⁶ is selected from the group consisting of hydrogen and amino,
R⁷ and R⁸ are independently of each other selected from the group consisting of hydrogen, amino and methyl, or together with the carbon atoms to which they are bonded, form a saturated or unsaturated five or six-membered ring,
R⁹ is selected from the group consisting of hydrogen, phenyl, branched or unbranched, saturated alkyl having 1 to 4 carbon atoms and branched or unbranched, saturated aminoalkyl having 1 to 4 carbon atoms, and
R¹¹ is a bond or hydrogen,
wherein the moiety of formula I-3 is covalently bonded to the Photoremovable Protecting Group Moiety B via a free bond of a nitrogen atom present in the moiety of formula I-3;
and
a moiety which is selected from the group consisting of the moieties of formula I-4, formula I-5 and formula I-6: 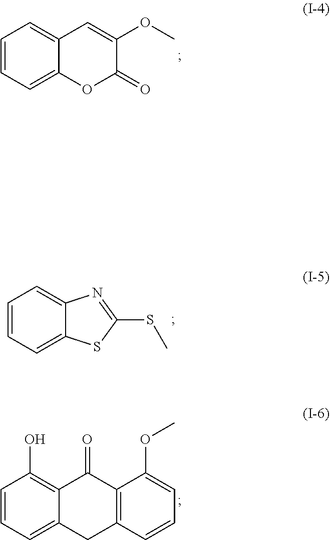 [SMILES: COc1cc2ccccc2oc1=O ; CSc1nc2ccccc2s1 ; COc1cccc2c1C(=O)c1c(O)cccc1C2]
wherein the moieties of formula I-4 and of formula I-6 are attached to the Photoremovable Protecting Group Moiety B via the respective free bond of an oxygen atom, and wherein the moiety of formula I-5 is attached to the Photoremovable Protecting Group Moiety B via the free bond of a sulfur atom;
and wherein
the Photoremovable Protecting Group Moiety B is a moiety of formula II 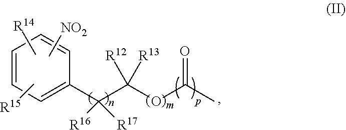
wherein
R¹² is selected from the group consisting of hydrogen and branched or unbranched, saturated alkyl with 1 to 4 carbon atoms;
R¹³ is selected from the group consisting of hydrogen, branched or unbranched, saturated alkyl with 1 to 4 carbon atoms and carboxy;
R¹⁴ and R¹⁵ are attached to the phenyl ring, wherein
R¹⁴ and R¹⁵ are both independently of each other selected from the group consisting of hydrogen, methoxy, carboxy, nitro and —O(CH₂)_{I}COOH, wherein 1 is an integer in the range from 1 to 3;
or
R¹⁴ and R¹⁵ together form an acetal group having two oxygen atoms attached to adjacent carbon atoms of the phenyl ring and having 1 to 3 carbon atoms outside the phenyl ring;
R¹⁶ and R¹¹ are hydrogen or, together with the carbon atom to which they are bonded, form a carbonyl group; and
m, n, and p are independently of each other 0 or 1, wherein preferably n=0 and wherein the Photoremovable Protecting Group Moiety B of formula II is attached to an organic MALDI Matrix Compound Moiety A via the free bond next to the letter “p” shown in formula II above.

2. Photo-caged MALDI Matrix Compound according to claim 1, wherein
the organic MALDI Matrix Compound Moiety A is a moiety selected from the group consisting of
a moiety of formula I-1 as defined in claim 1,
a moiety of formula I-2 as defined in claim 1
and
a moiety of formula I-3a 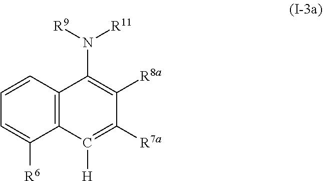
wherein
R⁶, R⁹ and R¹¹ have the meanings as defined in claim 1 for the moiety of formula I-3,
R^{7a} and R^{8a} are independently of each other selected from the group consisting of hydrogen and methyl,
and
wherein the moiety of formula I-3a is covalently bonded to the Photoremovable Protecting Group Moiety B via a free bond of a nitrogen atom present in the moiety 1-3a.

3. Photo-caged MALDI Matrix Compound according to claim 1, selected from the group consisting of:
a compound of formula III-1 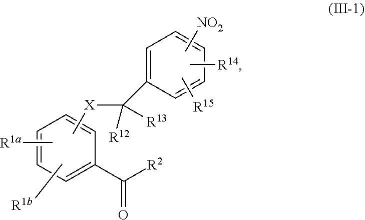
wherein
R^{1a}, R^{1b}, R² and X have the meanings as defined in claim 1 for the moiety of formula I-1,
and
R¹², R¹³, R¹⁴ and R¹⁵ have the meanings as defined in claim 1 for the moiety of formula II;
a compound of formula III-2 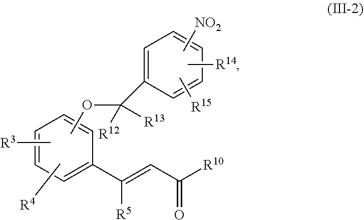
wherein
R³, R⁴, R⁵ and R¹⁰ have the meanings as defined in claim 1 for the moiety of formula I-2
and
R¹², R¹³, R¹⁴ and R¹⁵ have the meanings as defined in claim 1 for the moiety of formula II,
and
a compound of formula III-3 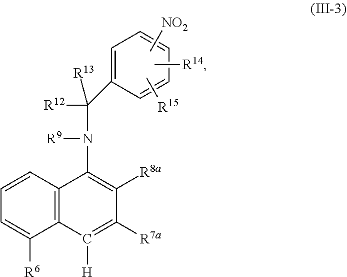
wherein
R⁶ and R⁹ have the meanings as defined in claim 1 for the moiety of formula I-3,
R¹², R¹³, R¹⁴ and R¹⁵ have the meanings as defined in claim 1 for the moiety of formula II,
and
R^{7a} and R^{8a} are independently of each other selected from the group consisting of hydrogen and methyl.

4. Photo-caged MALDI Matrix Compound according to claim 1, which is a compound of formula 1.1

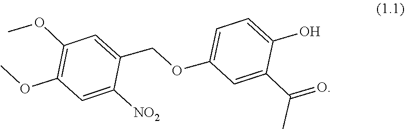 [SMILES: COc1cc(COc2ccc(O)c(C(C)=O)c2)c([N+](=O)[O-])cc1OC]

5. Sprayable liquid composition comprising a Photo-caged MALDI Matrix Compound according to claim 1.

6. Matrix-assisted laser desorption/ionization mass spectrometry method, comprising the steps:
S0) providing or preparing a Photo-caged MALDI Matrix Compound as defined in claim 1 or a sprayable liquid composition comprising the photo-caged MALDI matrix compound claim 1,
S1) combining the Photo-caged MALDI Matrix Compound or the sprayable liquid composition thereof as provided or prepared in step S0) with one or more analytes so that a Photo-sensitive MALDI Matrix Composite results,
S2) irradiating, in one, two, or more steps, the Photo-sensitive MALDI Matrix Composite resulting from step S1) so that
(i) the Photo-caged MALDI Matrix Compound is photo-cleaved,
and
(ii) one or more of said analytes are desorbed/ionized,
and
S3) analyzing one or more of said analytes desorbed/ionized in step S2) by mass spectrometry.

7. Method according to claim 6, wherein the Photo-sensitive MALDI Matrix Composite resulting from step S1) is kept under vacuum for a time period of at least one hour before it is irradiated in step S2),
wherein preferably
the Photo-sensitive MALDI Matrix Composite resulting from step S1) is kept under vacuum for a time period of at least 8 hours, before it is irradiated in step S2);
or
the Photo-sensitive MALDI Matrix Composite resulting from step S1) is kept under vacuum for a time period of from ≥36 hrs to ≤72 hrs, before it is irradiated in step S2);
and/or
the Photo-sensitive MALDI Matrix Composite resulting from step S1) is kept under vacuum at a pressure of 3300 Pa or below, before it is irradiated in step S2).

8. Method according to claim 6, wherein
step S2) and/or step S3) is conducted under vacuum, preferably at a pressure of 3300 Pa or below;
and/or
irradiating the Photo-sensitive MALDI Matrix Composite in step S2) comprises irradiating with a laser source.

9. Method according to claim 6, wherein
the method is a high-throughput method analyzing 64 or more samples per hour, wherein preferably irradiating the Photo-sensitive MALDI Matrix Composite in step S2) comprises successively irradiating all of said samples with the same laser source;
and/or
the matrix-assisted laser desorption/ionization mass spectrometry method is a mass spectrometry imaging method, wherein preferably in step S1) the Photo-caged MALDI Matrix Compound is combined with one or more analytes which are present in a tissue sample by contacting a liquid composition of the Photo-caged MALDI Matrix Compound.

10. Method according to claim 6, wherein step S1) comprises
co-crystallizing the Photo-caged MALDI Matrix Compound with the one or at least one of the more than one analyte;
and/or
contacting a liquid composition of a Photo-caged MALDI Matrix Compound, with a tissue sample so that one or more analytes from the tissue sample are combined with the Photo-caged MALDI Matrix Compound.

11. Method according to claim 6, wherein
the one or at least one of the more than one analyte is selected from the group consisting of proteins, peptides, lipids, nucleic acids, polysaccharides, glycopeptides, organometallic compounds and organic polymers;
and/or
the total mass ratio of the total mass of Photo-caged MALDI Matrix Compound present in the Photo-sensitive MALDI Matrix Composite: total mass of analyte present in the Photo-sensitive MALDI Matrix Composite is in the range of from ≥100:1 to ≤20 000:1.

12. Photo-sensitive MALDI Matrix Composite for use in a method of matrix-assisted laser desorption/ionization mass spectrometry, comprising or consisting of
C1) a matrix, completely or partially constituted by a Photo-caged MALDI Matrix Compound as defined in claim 1,
and embedded in said matrix
C2) one or more than one analyte, to be analyzed in the method of matrix-assisted laser desorption/ionization mass spectrometry.

13. Photo-sensitive MALDI Matrix Composite according to claim 12, wherein
the one or at least one of the more than one analyte is selected from the group consisting of proteins, peptides, lipids, nucleic acids, polysaccharides, glycopeptides, organometallic compounds and organic polymers;
and/or
the total mass ratio of the total mass of Photo-caged MALDI Matrix Compound present in the Photo-sensitive MALDI Matrix Composite: total mass of analyte present in the Photo-sensitive MALDI Matrix Composite is in the range of from ≥100:1 to ≤20 000:1.

14. Use of a Photo-caged MALDI Matrix Compound as defined in claim 1
as matrix compound in a method of matrix-assisted laser desorption/ionization mass spectrometry
and/or
for preparing a Photo-sensitive MALDI Matrix Composite comprising or consisting of:
C1) a matrix, completely or partially constituted by the Photo-caged MALDI Matrix Compound,
and embedded in said matrix
C2) one or more than one analyte, to be analyzed in the method of matrix-assisted laser desorption/ionization mass spectrometry.

15. Method of identifying for a given type of analyte and predetermined vacuum conditions a suitable Photo-caged MALDI Matrix Compound according to claim 1, comprising the steps of
providing for said type of analyte a suitable uncaged MALDI matrix compound comprising the organic MALDI Matrix Compound Moiety A but not comprising the Photoremovable Protecting Group Moiety B,
preparing or providing one or more derivatives of said suitable uncaged MALDI matrix compound, wherein each of said derivatives is a different Photo-caged MALDI Matrix Compound comprising a different Photoremovable Protecting Group Moiety B which is covalently bonded to the organic MALDI Matrix Compound Moiety A present in said uncaged MALDI matrix compound,
assessing the dependency of MALDI analysis results from predetermined vacuum conditions for the combinations of said type of analyte with both said suitable uncaged MALDI matrix compound and said one or more derivatives of said suitable uncaged MALDI matrix compound,
and
identifying the suitable Photo-caged MALDI Matrix Compound comprising a Photoremovable Protecting Group Moiety B which is covalently bonded to an organic MALDI Matrix Compound Moiety A by comparing the results of the assessments.
